SEQUENCE LISTING (7 sequences)

SEQ ID NO: 1 - protein, 31 aa, Homo sapiens
HAEGTFTSDVSSYLEGQAAKEFIAWLVKGRG

SEQ ID NO: 2 - protein, 39 aa, Heloderma suspectum
HGEGTFTSDLSKQMEEEAVRLFIEWLKNGGPSSGAPPPS

SEQ ID NO: 3 - protein, 39 aa, Heloderma suspectum
HSDGTFTSDLSKQMEEEAVRLFIEWLKNGGPSSGAPPPS

SEQ ID NO: 4 - protein, 33 aa, Artificial Sequence
  Synthetic
  MISC_FEATURE (1)..(1): This residue is L-His, imidazopropionyl, alpha-hydroxy-histidine, D-His, desamino-His, 2-amino-His, beta-hydroxy-His, homo-His, N-alpha-acetyl-His, N-alpha-formyl-His, alpha-fluoromethyl-His, alpha-methyl-His, 3-pyridyl-Ala, 2-pyridyl-Ala, or 4-pyridyl-Ala
  MISC_FEATURE (2)..(2): This residue is Ala, Gly, Val, Leu, Ile, Thr, Ser, Lys, Aib, (1-aminocyclopropyl) carboxylic acid, (1-aminocyclobutyl) carboxylic acid (CA), (1-aminocyclopentyl) CA, (1-aminocyclohexyl) CA, (1-aminocycloheptyl) CA, or
  MISC_FEATURE (6)..(6): This residue is Lys or Phe
  MISC_FEATURE (10)..(10): This residue is Val or Leu
  MISC_FEATURE (12)..(12): This residue is Ser, Arg, Asn, Gln, or Glu
  MISC_FEATURE (13)..(13): This residue is Tyr or Gln
  MISC_FEATURE (14)..(14): This residue is Leu, Lys, or Met
  MISC_FEATURE (16)..(16): This residue is Gly, Glu, Lys, or Aib
  MISC_FEATURE (17)..(17): This residue is Gln, Glu, or Arg
  MISC_FEATURE (18)..(18): This residue is Ala or Lys
  MISC_FEATURE (19)..(19): This residue is Ala or Val
  MISC_FEATURE (20)..(20): This residue is Val, His, Lys or Arg
  MISC_FEATURE (24)..(24): This residue is Ala, Glu, or Arg
  MISC_FEATURE (25)..(25): This residue is Trp or His
  MISC_FEATURE (28)..(28): This residue is Glu, Asn, Gly, Gln, or Arg
  MISC_FEATURE (29)..(29): This residue is Gly, Aib, or absent
  MISC_FEATURE (30)..(30): This residue is Arg, Gly, Lys, or absent
  MISC_FEATURE (31)..(31): This residue is Gly, Ala, Glu, Pro, Lys, Arg, or absent
  MISC_FEATURE (32)..(32): This residue is Ser, Gly, Ala, Glu, Gln, Pro, Arg, or absent
  MISC_FEATURE (33)..(33): This residue is Gly or absent
XXEGTXTSDXSXXXEXXXXXKFIXXLVXXXXXX

SEQ ID NO: 5 - protein, 29 aa, Heloderma suspectum
HGEGTFTSDLSKQMEEEAVRLFIEWLKNG

SEQ ID NO: 6 - protein, 29 aa, Homo sapiens
HAEGTFTSDVSSYLEGQAAKEFIAWLVKG

SEQ ID NO: 7 - protein, 31 aa, Artificial Sequence
  Synthetic
  MISC_FEATURE (1)..(1): This residue is L-His, (S)-2-Hydroxy-3-(1H-imidazol-4-yl)-propionic acid, D-His, desamino-His, Nalpha-acetyl-His, or Nalpha-formyl-His
  MISC_FEATURE (2)..(2): This residue is Ala, Ser, Aib, (1-aminocyclopropyl) carboxylic acid, or (1-aminocyclobutyl) carboxylic acid
  MISC_FEATURE (10)..(10): This residue is Val or Leu
  MISC_FEATURE (12)..(12): This residue is Ser or Arg
  MISC_FEATURE (13)..(13): This residue is Tyr or Gln
  MISC_FEATURE (14)..(14): This residue is Leu or Met
  MISC_FEATURE (16)..(16): This residue is Gly or Glu
  MISC_FEATURE (17)..(17): This residue is Gln, Glu, or Arg
  MISC_FEATURE (19)..(19): This residue is Ala or Val
  MISC_FEATURE (20)..(20): This residue is Arg or Lys
  MISC_FEATURE (21)..(21): This residue is Glu or Leu
  MISC_FEATURE (24)..(24): This residue is Ala or Glu
  MISC_FEATURE (25)..(25): This residue is Trp or His
  MISC_FEATURE (27)..(27): This residue is Val or Arg
  MISC_FEATURE (28)..(28): This residue is Arg, Lys, His, Asn, or Gln
  MISC_FEATURE (29)..(29): This residue is Gly or Aib
XXEGTFTSDXSXXXEXXAXXXFIXXLXXXKK

(19) United States
(12) Patent Application Publication
(10) Pub. No.: US 2018/0263915 A1
(43) Pub. Date: Sep. 20, 2018

(54) Pharmaceutical Composition for Oral GLP-1 Administration Comprising a Tablet Core and Immediate Release Coating

(71) Applicant: Novo Nordisk A/S, Bagsvaerd (DK)

(72) Inventors: Pernille Nybo, Roedovre (DK); Flemming S. Nielsen, Roskilde (DK); Birgitte Nissen, Glostrup (DK); Per Sauerberg, Farum (DK); Rikke B. Skyggebjerg, Copenhagen V (DK)

(21) Appl. No.: 15/546,334

(22) PCT Filed: Jan. 28, 2016
(86) PCT No.: PCT/EP2016/051797

§ 371 Date: Jul. 26, 2017

(30) Foreign Application Priority Data
Jan. 29, 2015 (EP) 15153049.0
Apr. 7, 2015 (EP) 15162529.0
Apr. 7, 2015 (EP) PCT/EP2015/057442
Oct. 7, 2015 (EP) 15188730.4

(51) Int. Cl. A61K 9/28; A61K 9/20; A61K 38/26; A61K 38/22; A61K 9/00

(52) U.S. Cl. CPC A61K 9/284; A61K 9/2013; A61K 9/2893; A61K 38/2278; A61K 9/0053; A61K 38/26

(57) ABSTRACT

The present invention relates to pharmaceutical compositions comprising a tablet core and an immediate release coating, wherein said tablet core comprises a GLP-1 peptide and an absorption enhancer, as well as uses thereof.

### FIELD OF THE INVENTION

The present invention relates to pharmaceutical compositions comprising a tablet core and an immediate release coating, wherein said tablet core comprises a GLP-1 peptide and an absorption enhancer, as well as uses thereof.

### INCORPORATION-BY-REFERENCE OF THE SEQUENCE LISTING

The Sequence Listing, entitled “SEQUENCE LISTING”, is 6.239 bytes, was created on 27 Jan. 2016, and is incorporated herein by reference.

### BACKGROUND

Many pathological states due to deficiencies in or complete failure of the production of certain macromolecules (e.g. proteins and peptides) are treated with an invasive and inconvenient parenteral administration of therapeutic macromolecules. One example hereof is the administration of GLP-1 peptides in the treatment type 2 diabetes. The oral route is desirable for administration due to its non-invasive nature and has a great potential to decrease the patient's discomfort related to drug administration and to increased drug compliance. However, several barriers exist; such as the enzymatic degradation in the gastrointestinal (GI) tract and limited permeability over the gastrointestinal membrane leading to insufficient and variable absorption. Until now, there are no products for oral delivery of GLP-1 to be marketed.

Provision of a solid oral dosage form which would facilitate the administration of GLP-1 is desirable. The advantages of solid oral dosage forms over other dosage forms include ease of manufacture, storage and administration. There may also be advantages relating to convenience of administration increasing patient compliance.

Obtaining an acceptable bioavailability of a peptide after administration thereof via the oral route is not easy. An oral composition suitable for the treatment of patients with an GLP-1 peptide, where an effective bioavailability of said GLP-1 peptide is obtained, is thus needed.

### SUMMARY

The present invention relates to solid oral GLP-1 compositions consisting of a tablet core and an immediate release coating, wherein said tablet core comprises an absorption enhancer, wherein said absorption enhancer may be a salt of a medium-chain fatty acid.

In one embodiment of the invention, the immediate release coating of pharmaceutical compositions of the present invention is a polymer based coating, selected from a polyvinyl alcohol (PVA) based coating and a hydroxypropylmethylcellulose (HPMC) based coating.

In one embodiment, the pharmaceutical composition of the present invention comprises an immediate release coating which dissolves in aqueous medium at any pH.

In one embodiment of the invention, the immediate release coating comprises about 25-55% polyvinyl alcohol. In another one embodiment of the invention, the immediate release coating comprises hydroxypropylmethylcellulose.

In one embodiment, the pharmaceutical composition of the present invention comprises a tablet core comprising sodium caprate, sorbitol, stearic acid and GLP-1 receptor agonist.

In one embodiment, the pharmaceutical composition of the present invention is for use as a medicament. In one embodiment, the pharmaceutical composition of the present invention is for use as a medicament for the treatment of type 1 and type 2 diabetes or obesity.

In one embodiment, the pharmaceutical composition is for oral administration.

In one embodiment, the pharmaceutical composition of the present invention is in the form of a tablet, a capsule or a mini-tablet.

In another embodiment, the present invention provides methods for producing a pharmaceutical composition according to the present invention. In one embodiment a method for producing a pharmaceutical composition of the invention comprises the steps of preparing a tablet core and directly coating said immediate release coating on the outer surface of the tablet core.

In another embodiment, the invention may also solve further problems that will be apparent from the disclosure of the exemplary embodiments.

### DESCRIPTION

The present invention provides pharmaceutical compositions comprising a tablet core and an immediate release coating, wherein said tablet core comprises a GLP-1 peptide and a salt of a medium-chain fatty acid, as well as uses thereof.

The present invention provides a pharmaceutical composition comprising a GLP-1 peptide, wherein said pharmaceutical composition is effective in providing a therapeutically effective amount of a GLP-1 peptide in a subject, when administered to said subject by oral administration.

In one embodiment, the pharmaceutical composition of the present invention is suitable for immediate release of a GLP-1 peptide.

In one embodiment, the present invention provides a pharmaceutical composition comprising a tablet core and an immediate release coating selected from a polyvinyl alcohol (PVA) based coating and a hydroxypropylmethylcellulose (HPMC) based coating, wherein said tablet core comprises a salt of a medium-chain fatty acid and an GLP peptide.

In one embodiment, the pharmaceutical compositions of the present invention composition consist of a tablet core and an immediate release coating, wherein said tablet core comprises a pharmaceutical active ingredient and an absorption enhancer, wherein said pharmaceutically active ingredient is a GLP-1 peptide and wherein said absorption enhancer is a sodium caprate.

In one embodiment a pharmaceutical composition of the present invention consists of a tablet core and an immediate release coating, wherein said tablet core comprises a pharmaceutical active ingredient and a salt of capric acid. i.e. the tablet core comprises a pharmaceutical active ingredient and sodium caprate.

In another embodiment a pharmaceutical composition of the present invention consists of a tablet core and an immediate release coating, wherein said tablet core comprises a GLP-peptide and a salt of capric acid, for example sodium caprate.

In one embodiment, the pharmaceutical compositions of the present invention comprise an immediate release coating which dissolves at all pH values.

The inventors have surprisingly found that a pharmaceutical composition according to the embodiments of the present invention is optimal for the administration of GLP-1 peptide by oral administration. The inventors have also surprisingly found that a pharmaceutical composition according to the present invention results in good bioavailability and a fast onset of action.

The inventors have surprisingly found that the composition according to the present invention is more effective for increasing bioavailability and decreasing Tmax for said GLP-1 peptide compared to a composition where the tablet according to the present invention was further coated by an enteric coating such as Acryl EZE® 930 (as sold by Colorcon® in 2013, PA, USA), exemplified in Example 2, Table 2.

### Coating

In one embodiment, the present invention provides a pharmaceutical composition comprising an immediate release coating. As used herein the term “immediate release coating” refers to a thin coating which dissolves independently of the pH of the surroundings.

In one embodiment an immediate release coating according to the invention is in the form of a film coating.

In one embodiment the immediate release coating is the outer layer of the pharmaceutical composition according to the present invention.

In one embodiment an immediate release coating according to the present invention comprises polymers that can be used in aqueous coating processes, wherein said immediate release coating is applied to the tablet core in an aqueous dispersion or solution.

In one embodiment an immediate release coating according to the invention is Opadry®. In one embodiment an immediate release coating according to the invention is Opadry® Clear. In one embodiment an immediate release coating according to the invention is Opadry®II. In one embodiment an immediate release coating according to the invention is Opadry®II Clear. In one embodiment an immediate release coating according to the invention is Opadry®II Pigmented. In one embodiment an immediate release coating according to the invention is Opadry®II Yellow.

In one embodiment a composition and/or an immediate release coating according to the present invention comprise excipients as known to the person skilled in the art. Non-limiting examples of such known excipients are disclosed in “Direct compression and the role of filler-binders” (p 173-217): by B. A. C. Carlin, in “Disintegrants in tabletting” (p 217-251): by R. C. Moreton, and in “Lubricants, glidants and adherents” (p 251-269), by N. A. Armstrong, in Pharmaceutical dosage forms: Tablets“, Informa Healthcare, N.Y., vol 2, 2008, L. L. Augsburger and S. W. Hoag”.

In one embodiment an immediate release coating of a composition according to the present invention is coated on to the surface of a tablet core according to the present invention in an amount of about 0-17.6 mg/cm2 relative to the tablet core. In one embodiment an immediate release coating of a composition according to the present invention is coated on to the outer surface of a tablet core according to the present invention in an amount in mg/cm2, relative to the tablet core, such as about 0, such as about 2.6, such as about 3.5, such as about 4.4, such as about 7.1, such as about 8, such as about 8.8, such as about 10.6, such as about 14.1 and such as about 17.6 mg/cm2.

In one embodiment an immediate release coating may be coated on top of a tablet core according to the present invention. In one embodiment immediate release coating may be coated on top of a tablet according to the present invention. In one embodiment an immediate release coating may be coated on top of the outer surface of a tablet core according to the present invention.

In one embodiment an immediate release coating material is dispersed or dissolved in aqueous medium, such as but not limited to water, resulting in immediate release coating dispersion. In one embodiment the immediate release coating material is dispersed in aqueous medium to form an immediate release coating dispersion for coating on top of a tablet or tablet core, where the immediate release coating material can form the immediate release coating or film. In one embodiment an immediate release coating dispersion is coated on top of a tablet core according to this invention. In one embodiment an immediate release coating dispersion is coated on top of a tablet according to this invention.

In one embodiment excipients are added to an immediate release coating dispersion. In one embodiment excipients are added to an immediate release coating dispersion in the amount of about 10% (w/w) of the total dry coating material in said immediate release coating dispersion. In one embodiment excipients are added to an immediate release coating dispersion in the amount of about 10% (w/w) of the total dry coating material in said immediate release coating dispersion, wherein said total dry coating material in said immediate release coating dispersion comprises an immediate release coating polymer as defined in the present invention.

In one embodiment, the present invention provides a pharmaceutical composition comprising an immediate release coating selected from a polyvinyl alcohol (PVA) based coating and a hydroxypropylmethylcellulose (HPMC) based coating.

In one embodiment, a polyvinyl alcohol and/or hydroxypropylmethylcellulose coating of the present invention is an OPADRY®II.

In one embodiment excipients are added to said immediate release coating dispersion in the amount of about 10% (w/w) of the total dry coating material in said immediate release coating dispersion, wherein said total dry coating material in said immediate release coating dispersion comprises immediate release coating polymer(s) such as comprised in Opadry®II from Colorcon®. In one embodiment excipients are added to said immediate release coating dispersion in the amount of about 10% (w/w) of the total dry coating material in said immediate release coating, wherein said total dry coating material in said immediate release coating dispersion comprises immediate release coating polymer(s) different from the one comprised in Opadry®II from Colorcon®. In one embodiment excipients are added to said immediate release coating dispersion in the amount of about 10% (w/w) of the total dry coating material in said immediate release coating, wherein said total dry coating material in said immediate release coating dispersion comprises an immediate release coating different from the one comprised in Opadry®II from Colorcon® and wherein said immediate release coating dissolves at any pH. In one embodiment excipients are added to said immediate release coating dispersion in the amount of about 10% (w/w) of the total dry coating material in said immediate release coating, wherein said total dry coating material in said immediate release coating dispersion comprises an immediate release coating different from the one comprised in Opadry®II from Colorcon® resulting in an immediate release coating.

### PVA

In one embodiment, the immediate release coating of the present invention comprises polyvinyl alcohol. In one embodiment a polyvinyl alcohol coating is an aqueous coating.

In one embodiment of the present invention, the immediate release coating comprises about 25-55% polyvinyl alcohol. In one embodiment of the present invention, the immediate release coating comprises about 38-46% (w/w) polyvinyl alcohol polymer.

In one embodiment a polyvinyl alcohol coating is an aqueous coating as disclosed in WO0104195A1.

In one embodiment a polyvinyl alcohol coating is an aqueous coating commercially available, comprising polyvinyl alcohol polymer, such as Opadry®II, Opadry® II Clear, Opadry® II Yellow and Opadry® II Pigmented.

In one embodiment a polyvinyl alcohol coating is an Opadry®II. As used herein the term “Opadry®II” refers to a composition comprising polyvinyl alcohol polymer and may be clear or pigmented. A product suitable to prepare a coating comprising Opadry®II may be obtained from Colorcon®, PA, USA, as sold in 2014.

In one embodiment a polyvinyl alcohol coating comprises Opadry® II Clear. As used herein the term “Opadry® II Clear” refers to a composition comprising polyvinyl alcohol polymer. A product suitable to prepare a coating comprising Opadry® II Clear may be obtained from Colorcon®, PA, USA, in the form of the product sold as product code 85F19250 in 2014.

In one embodiment a polyvinyl alcohol coating comprises Opadry® II Pigmented. As used herein the term “Opadry® II Pigmented” refers to a composition comprising polyvinyl alcohol polymer. A product suitable to prepare a coating comprising Opadry® II Pigmented may be obtained from Colorcon®, PA, USA, as sold in 2014.

In one embodiment Opadry® II Pigmented may be OPADRY®II Yellow. In one embodiment a polyvinyl alcohol coating comprises OPADRY®II Yellow. As used herein the term “Opadry®II Yellow” refers to a composition comprising polyvinyl alcohol polymer. A product suitable to prepare a coating comprising Opadry®II Yellow may be obtained from Colorcon®, PA, USA, in the form of the product sold as product code 85F32410 in 2014.

In one embodiment a polyvinyl alcohol based coating according to the present invention is an immediate release coating. In one embodiment a polyvinyl alcohol based coating is an aqueous coating. In one embodiment a polyvinyl alcohol based coating according to the present invention dissolves at all pH values.

### HPMC

In one embodiment, the immediate release coating of the present invention comprises hydroxypropylmethylcellulose polymer. In one embodiment a hydroxypropylmethylcellulose polymer coating is an aqueous coating.

In one embodiment a hydroxypropylmethylcellulose based coating is Opadry®. As used herein the term “Opadry®” refers to a composition comprising hydroxypropylmethylcellulose. A product suitable to prepare a coating comprising Opadry® may be obtained from Colorcon®, PA, USA, as sold in 2014.

In one embodiment a hydroxypropylmethylcellulose based coating is Opadry® Clear. As used herein the term “Opadry® Clear” refers to a composition comprising hydroxypropylmethylcellulose. A product suitable to prepare a coating comprising Opadry® Clear may be obtained from Colorcon®, PA, USA, in the form of the product sold as product code 03K19229 in 2014.

In one embodiment, the immediate release coating of the present invention comprises hydroxypropylmethylcellulose. In one embodiment a hydroxypropylmethylcellulose based coating is an aqueous coating. In one embodiment a hydroxypropylmethylcellulose based coating according to the present invention dissolves at all pH values.

### Tablet Core

In one embodiment, the present invention relates to a pharmaceutical composition consisting of a tablet core and an immediate release coating, wherein said tablet core comprises a pharmaceutical active ingredient and an absorption enhancer.

In one embodiment a tablet core of a composition according to the present invention is a tablet, a capsule or a mini-tablet.

### Absorption Enhancer

The solid pharmaceutical composition comprises an absorption enhancer. The absorption enhancer may comprise a salt of a medium-chain fatty acid. As used herein the term medium-chain fatty acid refers to a saturated fatty acid consisting of 6-14 carbon atoms, such as 8-12 carbon atoms. The absorption enhancer may be a salt of capric acid. Capric acid may also be referred to as decanoic acid (CH₃(CH₂)₈COOH). The salt of capric acid may be sodium caprate (i.e. CH₃(CH₂)₈COONa). The solid pharmaceutical composition may comprise a salt of capric acid.

In one embodiment the salt of capric acid comprised in the present invention is in the form of a sodium salt. In another embodiment, the salt of capric acid is sodium caprate. The term “sodium caprate” as used herein means sodium salt of capric acid.

In one embodiment a tablet core according to the present invention comprises a salt of a medium-chain fatty acid in the amount of about 50-700 mg. In one embodiment a tablet core according to the present invention comprises a salt of a medium-chain fatty acid in the amount of about 50-550 mg. In one embodiment a tablet core according to the present invention comprises a salt of a medium-chain fatty acid in the amount of about 50-700 mg. In one embodiment a tablet core according to the present invention comprises a salt of a medium-chain fatty acid in the amount of about 150-600 mg. In one embodiment a tablet core according to the present invention comprises a salt of a medium-chain fatty acid in the amount of about 150-600 mg. In one embodiment a tablet core according to the present invention comprises a salt of a medium-chain fatty acid in the amount of about 180-550 mg. In one embodiment a tablet core according to the present invention comprises a salt of a medium-chain fatty acid in the amount of about 180 mg. In one embodiment a tablet core according to the present invention comprises a salt of a medium-chain fatty acid in the amount of about 400 mg. In one embodiment a tablet core according to the present invention comprises a salt of a medium-chain fatty acid in the amount of about 550 mg. In one embodiment a tablet core according to the present invention comprises a salt of a medium-chain fatty acid in the amount of up to about 700 mg. The salt of a medium-chain fatty acid may be a salt of capric acid. The salt of capric acid may be sodium salt of capric acid, also named sodium caprate.

In one embodiment of the present invention the layer and/or tablet core that is coated with and thus in contact with an immediate release coating according to this invention comprises a salt of capric acid, in an amount of about 50-700 mg. In one embodiment of the present invention the layer and/or tablet core that is coated with and thus in contact with an immediate release coating according to this invention comprises a salt of capric acid, in an amount of about 50-550 mg. In one embodiment of the present invention the layer and/or tablet core that is coated with and thus in contact with an immediate release coating according to this invention comprises a salt of capric acid, in an amount of about 180-550 mg. In one embodiment of the present invention the layer and/or tablet core that is coated with and thus in contact with an immediate release coating according to this invention comprises about 50 mg or more, such as about 150 mg or more, such as about 180 mg or more, such as about 400 mg or more sodium caprate.

In some embodiments, the invention relates to a pharmaceutical composition, e.g. in the form of a tablet, comprising a GLP-1 receptor agonist as described herein and an absorption enhancer selected from SNAC and sodium caprate. SNAC is the sodium salt of N-(8-(2-hydroxybenzoyl)amino)caprylic acid and may be prepared using the method described in e.g. WO96/030036, WO00/046182, WO01/092206 or WO2008/028859.

### GLP-1 Peptide in the Core

In one embodiment a tablet core according to the present invention comprises a pharmaceutical active ingredient. In one embodiment a tablet core according to the present invention comprises a GLP-1 receptor agonist. In one embodiment a tablet core according to the present invention comprises a GLP-1 receptor agonist selected from the examples of WO2006/097537, WO2011/080103 and WO2012/140117.

In one embodiment, the GLP-1 peptide is selected from the group consisting of:
N-{Epsilon-26}-[[2-(2-[2-(2-[2-(2-[4-(17-Carboxyheptadecanoylamino)-4(S)-carboxybutyrylamino]ethoxy)ethoxy]acetylamino)ethoxy]ethoxy)acetyl][Aib8,Arg34]GLP-1-(7-37)peptide.

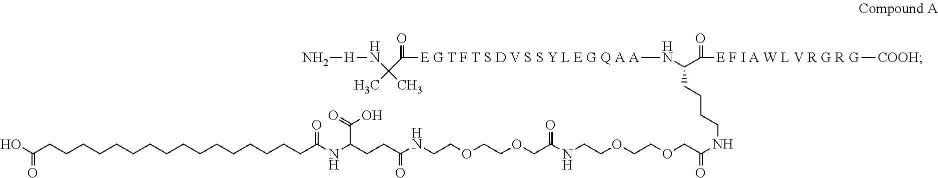

N-{Epsilon-26}-[(2-[2-(2-{2-[2-(2-{(S)-4-Carboxy-4-[10-(4-carboxyphenoxy)decanoylamino]butyrylamino}ethoxy)ethoxy]acetylamino}ethoxy)ethoxy]acetyl}, N-{Epsilon-37}-{2-[2-(2-{2-[2-(2-{(S)-4-carboxy-4-[10-(4-carboxyphenoxy)decanoylamino]butyrylamino}ethoxy)ethoxy]acetylamino}ethoxy)ethoxy]acetyl}-[Aib8,Arg34, Lys37]GLP-1(7-37)-peptide

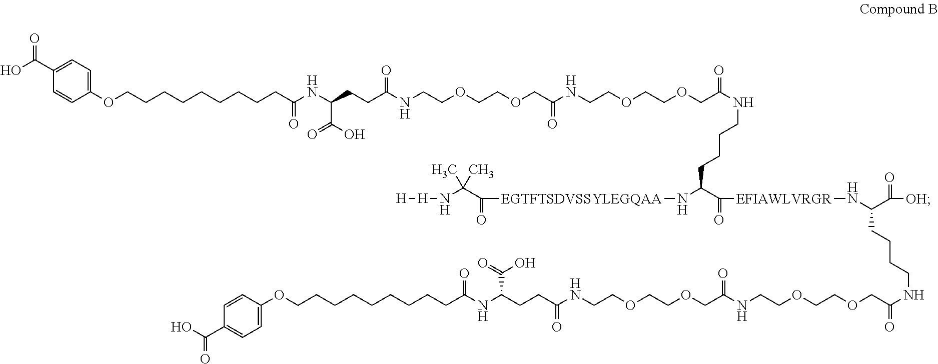

N-{Epsilon-27}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl], N-{Epsilon-36}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib8, Glu22,Arg26, Lys27, Glu30,Arg34, Lys36]-GLP-1-(7-37)-peptidyl-Glu-Gly

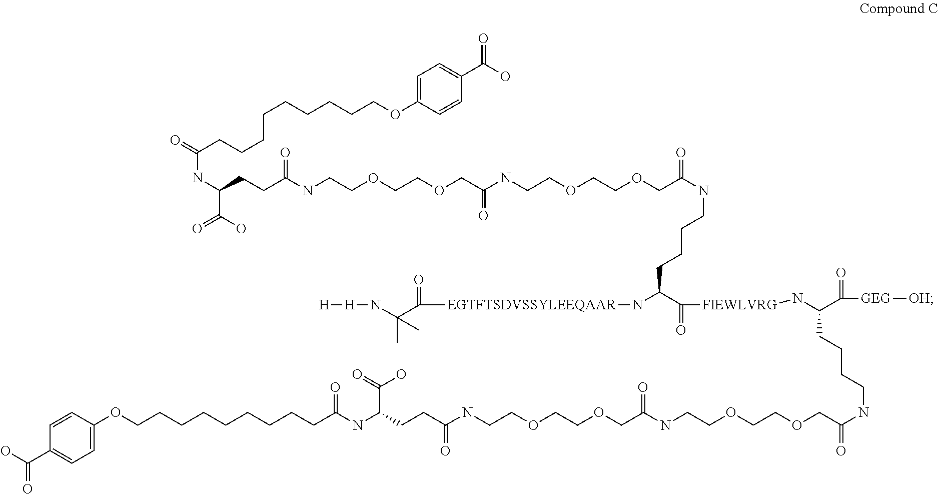

N{Epsilon-36}-[2-[2-[2-[[2-[2-[2-[[(4S)-4-carboxy-4-[1-(4-carboxyphenoxy)undecanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl]-[Aib8,Glu22,Arg26,Arg34,Lys36,Lys37]-GLP-1-(7-37)-peptide

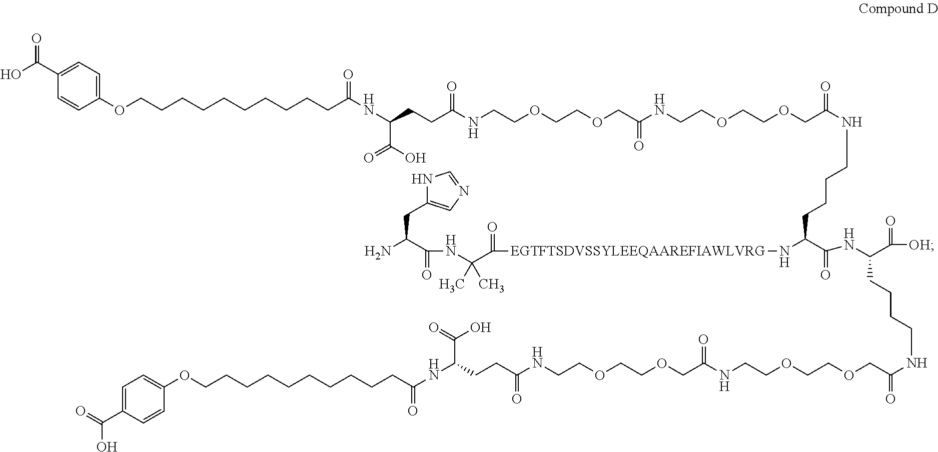

and
N{Epsilon-36}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl]-[Aib8,Arg26,Arg34,Lys36,Lys37]-GLP-1-(7-37)-peptide

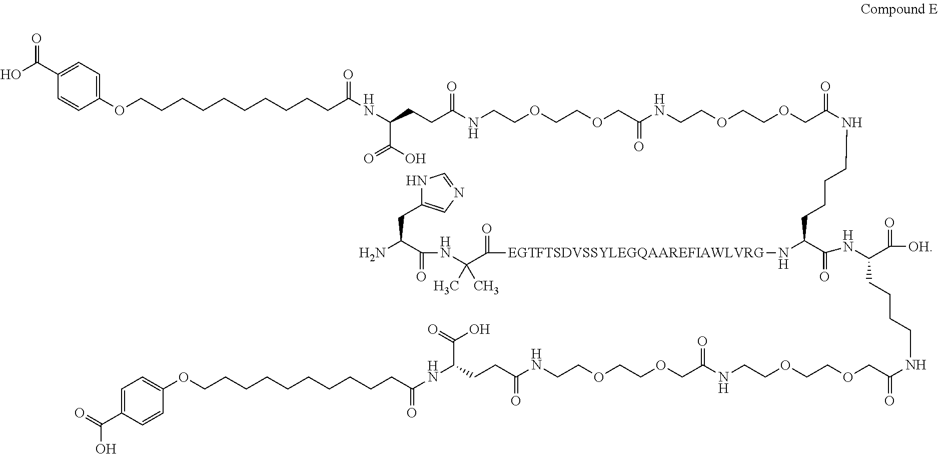

Compound C may also be illustrated as follows

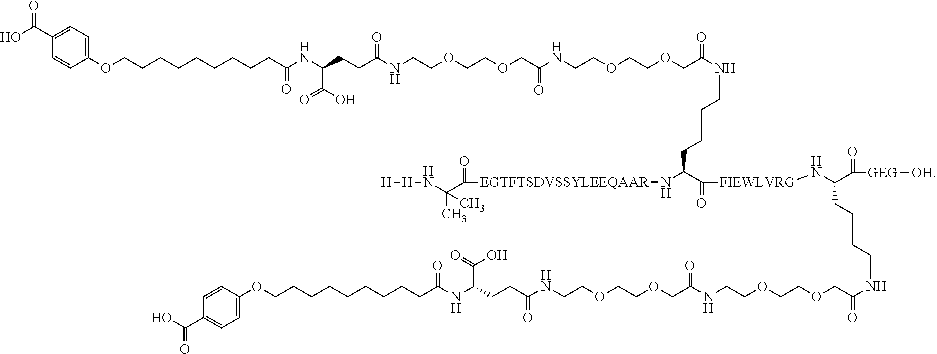

In one embodiment, the GLP-1 peptide is
N{Epsilon-36}-[2-[2-[2-[[2-[2-[2-[[(4S)-4-carboxy-4-[10-(3-carboxyphenoxy)decanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[10-(3-carboxyphenoxy)decanoylamino]butanoyl]amino]ethoxy]ethoxy]acetyl]amino]ethoxy]ethoxy]acetyl]-[Aib8,Glu22,Arg26,Arg34,Lys36,Lys37]-GLP-1-(7-37)-peptide

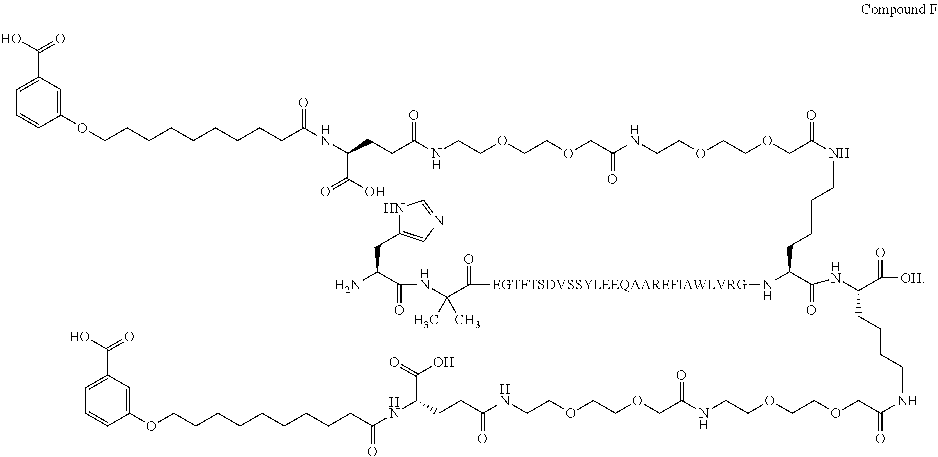

In one embodiment the GLP-1 peptide is selected from the group consisting of Compound A, Compound B, Compound C, Compound D, Compound E, and Compound F. In one embodiment the GLP-1 peptide is selected from the group consisting of Compound A, Compound B, Compound C, and Compound F. In one embodiment the GLP-1 peptide is selected from the group consisting of Compound A, Compound B and Compound C.

### GLP-1 Receptor Agonist

The pharmaceutical compositions of the present invention comprise a GLP-1 receptor agonist. The GLP-1 receptor agonist may be a GLP-1 peptide or an analogue or derivative thereof. The GLP-1 receptor agonist may be a derivative of a GLP-1 analogue. The GLP-1 receptor agonist may be human GLP-1, exendin-4 or an analogue or derivative thereof. The GLP-1 receptor agonist may be acylated. The GLP-1 receptor agonist may comprise a peptide comprising no more than 15 substitutions, deletions and/or additions of amino acids relative to human GLP-1 or exendin-4. In particular, the GLP-1 receptor agonist may comprise a peptide comprising no more than 10, such as no more than 9, no more than 8, no more than 7, no more than 6, no more than 5, or no more than 4, substitutions, deletions and/or additions of amino acids relative to human GLP-1 or exendin-4.

A receptor agonist may be defined as an analogue that binds to a receptor and elicits a response typical of the natural ligand. A full agonist may be defined as one that elicits a response of the same magnitude as the natural ligand (see e.g. “Principles of Biochemistry”, A L Lehninger, D L Nelson, M M Cox, Second Edition, Worth Publishers, 1993, page 763).

Thus, for example, a “GLP-1 peptide”, “GLP-1 agonist”, “GLP-1 receptor agonist”, “GLP-1 agonist” or “GLP-1 receptor agonist peptide” as used herein, is defined as a compound which is capable of binding to the GLP-1 receptor and capable of activating it.

### GLP-1 Peptides and Analogues

GLP-1 is an incretin hormone produced by the endocrine cells of the intestine following ingestion of food. GLP-1 is a regulator of glucose metabolism, and the secretion of insulin from the beta cells of the islets of Langerhans in the pancreas. GLP-1 also causes insulin secretion in the diabetic state. The half-life in vivo of GLP-1 itself is, however, very short, thus, ways of prolonging the half-life of GLP-1 in vivo has attracted much attention. WO 98/08871 discloses protracted GLP-1 analogues and derivatives based on human GLP-1(7-37) which have an extended half-life, including liraglutide, a GLP-1 derivative for once daily administration developed by Novo Nordisk A/S marketed as Victoza®, for the treatment of type 2 diabetes.

The term “GLP-1”, “GLP-1 peptide” or “hGLP-1” as used herein refers to the human Glucagon-Like Peptide-1 (GLP-1(7-37)), the sequence of which is included in the sequence listing as SEQ ID 1, or an analogue thereof. The peptide having the sequence of SEQ ID 1 may also be designated “native” GLP-1.

The Homo sapiens GLP-1(7-37) sequence is:

(SEQ ID 1)
HAEGTFTSDV SSYLEGQAAK EFIAWLVKGR G;

the Homo sapiens GLP-1(7-35) sequence is:

(SEQ ID 6)
HAEGTFTSDV SSYLEGQAAK EFIAWLVKG.

The term “GLP-1 analogue” or “analogue of GLP-1” as used herein refers to a peptide, or a compound, which is a variant of GLP-1(7-37) (SEQ ID 1) or of GLP-1(7-35) (SEQ ID 6).

In the sequence listing, the first amino acid residue (i.e. histidine) of SEQ ID 1 is assigned No. 1. However, in what follows—according to established practice in the art—this histidine residue is referred to as No. 7, and subsequent amino acid residues are numbered accordingly, ending with glycine No. 37. Therefore, generally, any reference herein to an amino acid residue number or a position number of the GLP-1(7-37) sequence is to the sequence starting with His at position 7 and ending with Gly at position 37.

For the purposes of numbering in Formula I, the same principle is used, i.e. start position X₇ corresponds to histidine in position 7 of native GLP-1 and ends in position X₃₇, corresponding to position 37 in native GLP-1(7-37) sequence.

The same principle applies for numbering of GLP-1(7-35): histidine residue is referred to as No. 7, and subsequent amino acid residues are numbered accordingly, ending with glycine No. 35.

GLP-1 analogues of the invention may be described by reference to i) the number of the amino acid residue in native GLP-1(7-37) or GLP-1(7-35), which corresponds to the amino acid residue which is changed (i.e., the corresponding position in native GLP-1), and to ii) the actual change.

In other words, a GLP-1 analogue is a GLP-1(7-37) or GLP-1(7-35) peptide in which a number of amino acid residues have been changed when compared to native GLP-1(7-37) (SEQ ID 1) or GLP-1(7-35) (SEQ ID 6). These changes may represent, independently, one or more amino acid substitutions, additions, and/or deletions.

The following are non-limiting examples of suitable analogue nomenclature.

Analogues “comprising” certain specified changes may comprise further changes, when compared to SEQ ID 1 or SEQ ID 6. In a particular embodiment, the analogue “has” the specified changes.

As is apparent from the above examples, amino acid residues may be identified by their full name, their one-letter code, and/or their three-letter code. These three ways are fully equivalent.

The expressions “a position equivalent to” or “corresponding position” may be used to characterise the site of change in a variant GLP-1(7-37) sequence by reference to native GLP-1(7-37) (SEQ ID 1) or GLP-1(7-35) (SEQ ID 6). Equivalent or corresponding positions, as well as the number of changes, are easily deduced, e.g. by simple handwriting and eyeballing; and/or a standard protein or peptide alignment program may be used, such as “align” which is based on a Needleman-Wunsch algorithm. This algorithm is described by Needleman, S. B. and Wunsch, C. D.; Journal of Molecular Biology 1970 48: 443-453; and the align program by Myers and W. Miller in “Optimal Alignments in Linear Space” CABIOS (computer applications in the biosciences) 1988 4 11-17. For the alignment, the default scoring matrix BLOSUM62 and the default identity matrix may be used, and the penalty for the first residue in a gap may be set at −12, or preferably at −10, and the penalties for additional residues in a gap at −2, or preferably at −0.5.

For an overview, GLP-1 receptor agonist peptides may be aligned as illustrated below:

| | |
|---|---|
| | 7 10 20 30 |
| GLP-1(7-35) | HAE GTFTSDVSSY LEGQAAKEFI AWLVKG |
| Exendin-4(1-29) | HGE GTFTSDLSKQ MEEEAVRLFI EWLKNG |
| Exendin-3(1-39) | HSD GTFTSDLSKQ MEEEAVRLFI EWLKNGGPSSGAPPPS |
| | 1 4 14 24 39 |

The term “GLP-1 peptide”, as e.g. used in the context of this invention, refers to a compound which comprises a series of amino acids interconnected by amide (or peptide) bonds.

A GLP-1 receptor agonist peptide of the invention may be any polypeptide comprising (i.e. including, but not limited to) an amino acid sequence as described herein, and thus may comprise additional amino acid residues.

In one embodiment the GLP-1 receptor agonist peptide of the invention comprise at least 31 amino acids.

In another embodiment, the GLP-1 receptor agonist peptide of the invention is composed of at least 32, or at least 33, or at least 34 amino acids.

In a third embodiment, the GLP-1 receptor agonist peptide of the invention holds of from 30 to 46 amino acid residues.

In a fourth embodiment, the GLP-1 receptor agonist peptide of the invention holds of from 32 to 42 amino acid residues.

In a fifth embodiment, the GLP-1 receptor agonist peptide of the invention holds of from 33 to 40 amino acid residues.

In a still further particular embodiment the GLP-1 receptor agonist peptide consists of amino acids interconnected by peptide bonds.

Amino acids are molecules containing an amine group and a carboxylic acid group, and, optionally, one or more additional groups, often referred to as a side chain.

The term “amino acid” includes proteinogenic amino acids (encoded by the genetic code, including natural amino acids, and standard amino acids), as well as non-proteinogenic (not found in proteins, and/or not coded for in the standard genetic code), and synthetic amino acids. Thus, the amino acids may be selected from the group of proteinogenic amino acids, non-proteinogenic amino acids, and/or synthetic amino acids.

Non-limiting examples of amino acids which are not encoded by the genetic code are gamma-carboxyglutamate, ornithine (Orn), norleucine (Nle) and phosphoserine. Non-limiting examples of synthetic amino acids are Aib (α-aminoisobutyric acid), β-alanine, and des-amino-histidine (alternative name imidazopropionic acid, abbreviated Imp).

In what follows, all amino acids of the GLP-1 peptide, for which the optical isomer is not stated, are to be understood to mean the L-isomer (unless otherwise specified).

The GLP-1 derivatives and analogues of the invention have GLP-1 activity. This term refers to the ability to bind to the GLP-1 receptor and initiate a signal transduction pathway resulting in insulinotropic action or other physiological effects as is known in the art.

In one embodiment the GLP-1 analogue comprises Formula I (SEQ ID 4):

Xaa7-Xaa8-Glu-Gly-Thr-Xaa12-Thr-Ser-Asp-Xaa16-Ser-Xaa18-Xaa19-Xaa20-Glu-Xaa22-Xaa23-Xaa24-Xaa25-Xaa26-Lys-Phe-Ile-Xaa30-Xaa31-Leu-Val-Xaa34-Xaa35-Xaa36-Xaa37-Xaa38-Xaa39, wherein Formula I:

Xaa7 is L-histidine, imidazopropionyl, α-hydroxy-histidine, D-histidine, desamino-histidine, 2-amino-histidine, β-hydroxy-histidine, homohistidine, Nα-acetyl-histidine, Nα-formyl-histidine, α-fluoromethyl-histidine, α-methyl-histidine, 3-pyridylalanine, 2-pyridylalanine, or 4-pyridylalanine;

Xaa8 is Ala, Gly, Val, Leu, Ile, Thr, Ser, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid;

Xaa12 is Lys or Phe;

Xaa16 is Val or Leu;

Xaa18 is Ser, Arg, Asn, Gln, or Glu;

Xaa19 is Tyr or Gln;

Xaa20 is Leu, Lys, or Met;

Xaa22 is Gly, Glu, Lys, or Aib;

Xaa23 is Gln, Glu, or Arg;

Xaa24 is Ala or Lys;

Xaa25 is Ala or Val;

Xaa26 is Val, His, Lys or Arg;

Xaa30 is Ala, Glu, or Arg;

Xaa31 is Trp or His;

Xaa34 is Glu, Asn, Gly, Gln, or Arg;

Xaa35 is Gly, Aib, or absent;

Xaa36 is Arg, Gly, Lys, or absent;

Xaa37 is Gly, Ala, Glu, Pro, Lys, Arg, or absent;

Xaa38 is Ser, Gly, Ala, Glu, Gln, Pro, Arg, or absent; and

Xaa39 is Gly or absent.

In one embodiment the GLP-1 analogue is a GLP-1 analogue of Formula I (SEQ ID 4). In one embodiment the GLP-1 analogue the peptide of Formula I is an analogue of GLP-1(7-37) (SEQ ID NO: 1). If Xaa38 of Formula I is absent, then Xaa39 of Formula I may also be absent. If Xaa37 of Formula I is absent, then Xaa38 and Xaa39 of Formula I may also be absent. If Xaa36 of Formula I is absent, then Xaa37, Xaa38, and Xaa39 of Formula I may also be absent. If Xaa35 of Formula I is absent, then Xaa36, Xaa37, Xaa38, and Xaa39 of Formula I may also be absent.

In one embodiment the GLP-1 analogue is a GLP-1 analogue of Formula I (SEQ ID 4), wherein Xaa7 is His; Xaa8 is Ala or Aib; Xaa12 is Lys or Phe; Xaa16 is Val; Xaa18 is Ser; Xaa19 is Tyr; Xaa20 is Leu or Lys; Xaa22 is Glu, Gly or Lys; Xaa23 is Glu or Gln; Xaa24 is Ala or Lys; Xaa25 is Ala or Val; Xaa26 is Lys or Arg; Xaa30 is Ala or Glu; Xaa31 is Trp or His; Xaa34 is Gly, Gln, or Arg; Xaa35 is Gly or absent; Xaa36 is Arg, Lys, or absent; Xaa37 is Gly, Lys, or absent; Xaa38 is Glu, Gln or absent; and Xaa39 is Gly or absent.

In one embodiment the GLP-1 analogue is a GLP-1 analogue of Formula I (SEQ ID 4), wherein Xaa7 is His; Xaa8 is Aib; Xaa12 is Phe; Xaa16 is Val; Xaa18 is Ser; Xaa19 is Tyr; Xaa20 is Leu; Xaa22 is Glu or Gly; Xaa23 is Gln; Xaa24 is Ala; Xaa25 is Ala; Xaa26 is Lys or Arg; Xaa30 is Ala or Glu; Xaa31 is Trp; Xaa34 is Arg; Xaa35 is Gly; Xaa36 is Arg or Lys; Xaa37 is Gly or Lys; Xaa38 is Glu or absent; and Xaa39 is Gly or absent.

### GLP-1 Derivatives

The term “derivative” as used herein in the context of a GLP-1 peptide means a chemically modified GLP-1 peptide, in which one or more substituents have been covalently attached to the peptide (also referred to herein as a “GLP-1 derivative”). The substituent may also be referred to as a side chain. Thus, the term “derivative” as used herein in the context of a GLP-1 analogue means a chemically modified GLP-1 analogue, in which one or more substituents have been covalently attached to the peptide. The GLP-1 derivative may comprise a GLP-1 peptide covalently attached by acylation to a substituent, wherein said substituent comprises a lipophilic moiety and optionally a distal aromatic group (e.g. 4-carboxyphenoxy).

In a particular embodiment, the side chain is capable of forming non-covalent aggregates with albumin, thereby promoting the circulation of the derivative with the blood stream, and also having the effect of protracting the time of action of the derivative, due to the fact that the aggregate of the GLP-1-derivative and albumin is only slowly disintegrated to release the drug substance. Thus, the substituent, or side chain, as a whole is preferably referred to as an albumin binding moiety.

In particular embodiments, the side chain has at least 10 carbon atoms, or at least 15, 20, 25, 30, 35, or at least 40 carbon atoms. In further particular embodiments, the side chain may further include at least 5 hetero atoms, in particular 0 and N, for example at least 7, 9, 10, 12, 15, 17, or at least 20 hetero atoms, such as at least 1, 2, or 3 N-atoms, and/or at least 3, 6, 9, 12, or 15 O-atoms.

In another particular embodiment the albumin binding moiety comprises a portion which is particularly relevant for the albumin binding and thereby the protraction, which portion may accordingly be referred to as a protracting moiety. The protracting moiety may be near, preferably at, the terminal (or distal, or free) end of the albumin binding moiety, relative to its point of attachment to the peptide.

In a still further particular embodiment the albumin binding moiety comprises a portion between the protracting moiety and the point of attachment to the peptide, which portion may be referred to as a linker, linker moiety, spacer, or the like. The linker may be optional, and hence in that case the albumin binding moiety may be identical to the protracting moiety.

In particular embodiments, the albumin binding moiety and/or the protracting moiety is lipophilic, and/or negatively charged at physiological pH (7.4).

The albumin binding moiety, the protracting moiety, or the linker may be covalently attached to a lysine residue of the GLP-1 peptide by acylation, i.e., via an amide bond formed between a carboxylic acid group thereof (of the albumin binding moiety, the protracting moiety, or the linker) and an amino group of the lysine residue. Additional or alternative conjugation chemistry includes alkylation, ester formation, or amide formation, or coupling to a cysteine residue, such as by maleimide or haloacetamide (such as bromo-/fluoro-/iodo-) coupling.

In a preferred embodiment, an active ester of the albumin binding moiety, preferably comprising a protracting moiety and a linker, is covalently linked to an amino group of a lysine residue, preferably the epsilon amino group thereof, under formation of an amide bond, as explained above.

Unless otherwise stated, when reference is made to an acylation of a lysine residue, it is understood to be to the epsilon-amino group thereof.

The term “fatty acid” refers to aliphatic monocarboxylic acids having from 4 to 28 carbon atoms, it is preferably un-branched, and it may be saturated or unsaturated.

The term “fatty diacid” refers to fatty acids as defined above but with an additional carboxylic acid group in the omega position. Thus, fatty diacids are dicarboxylic acids. The fatty diacid may comprise 14-22 carbon atoms. Non-limiting examples of fatty diacids are Chem C1 and Chem C2.

Each of the two linkers of the derivative of the invention may comprise the following first linker element:

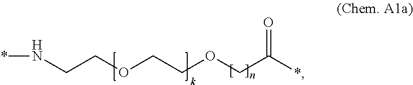

wherein k is an integer in the range of 1-5, and n is an integer in the range of 1-5.

In a particular embodiment, when k=1 and n=1, this linker element may be designated OEG, or a di-radical of 8-amino-3,6-dioxaoctanic acid, and/or it may be represented by the following formula:

*—NH—(CH₂)₂—O—(CH₂)₂—O—CH₂—CO—* (Chem. A2).

In another particular embodiment, each linker of the derivative of the invention may further comprise, independently, a second linker element, preferably a Glu di-radical, such as Chem. B1:

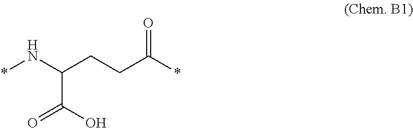 [SMILES: *NC(CCC(*)=O)C(=O)O]

wherein the Glu di-radical may be included p times, where p is an integer in the range of 1-3. Chem. B1 may also be referred to as gamma-Glu, or briefly gGlu, due to the fact that it is the gamma carboxy group of the amino acid glutamic acid which is here used for connection to another linker element, or to the epsilon-amino group of lysine. As explained above, the other linker element may, for example, be another Glu residue, or an OEG molecule. The amino group of Glu in turn forms an amide bond with the carboxy group of the protracting moiety, or with the carboxy group of, e.g., an OEG molecule, if present, or with the gamma-carboxy group of, e.g., another Glu, if present.

As explained above, the GLP-1 derivatives may be double-acylated, i.e. two albumin binding moieties are covalently attached to the GLP-1 peptide.

In a particular embodiment, the two albumin binding moieties (i.e. the entire side chains) are similar, preferably substantially identical, or, most preferably, identical.

In another particular embodiment, the two protracting moieties are similar, preferably substantially identical, or, most preferably, identical.

In a still further particular embodiment, the two linkers are similar, preferably substantially identical, or, most preferably identical.

The term “substantially identical” includes differences from identity which are due to formation of one or more salts, esters, and/or amides; preferably formation of one or more salts, methyl esters, and simple amides; more preferably formation of no more than two salts, methyl esters, and/or simple amides; even more preferably formation of no more than one salt, methyl ester, and/or simple amide; or most preferably formation of no more than one salt.

In the context of chemical compounds such as the albumin binding moieties, protracting moieties, and linkers, similarity and/or identity may be determined using any suitable computer program and/or algorithm known in the art.

For example, the similarity of two protracting moieties, two linkers, and/or two entire side chains may suitably be determined using molecular fingerprints. Fingerprints is a mathematical method of representing a chemical structure (see e.g. Chemoinformatics: A textbook, Johann Gasteiger and Thomas Engel (Eds), Wiley-VCH Verlag, 2003).

Examples of suitable fingerprints include, without limitation, UNITY fingerprints, MDL fingerprints, and/or ECFP fingerprints, such as ECFP_6 fingerprints (ECFP stands for extended-connectivity fingerprints).

In particular embodiments, the two protracting moieties, the two linkers, and/or the two entire side chains are represented as a) ECFP_6 fingerprints; b) UNITY fingerprints; and/or c) MDL fingerprints.

The Tanimoto coefficient is preferably used for calculating the similarity of the two fingerprints, whether a), b), or c) is used.

In particular embodiments, whether a), b), or c) is used, the two protracting moieties, the two linkers, and/or the two entire side chains, respectively, have a similarity of at least 0.5 (50%); preferably at least 0.6 (60%); more preferably at least 0.7 (70%), or at least 0.8 (80%); even more preferably at least 0.9 (90%); or most preferably at least 0.99 (99%), such as a similarity of 1.0 (100%).

UNITY fingerprints may be calculated using the programme SYBYL (available from Tripos, 1699 South Hanley Road, St. Louis, Mo. 63144-2319 USA). ECFP_6 and MDL fingerprints may be calculated using the programme Pipeline Pilot (available from Accelrys Inc., 10188 Telesis Court, Suite 100, San Diego, Calif. 92121, USA).

For more details, see for example J. Chem. Inf. Model. 2008, 48, 542-549; J. Chem. Inf. Comput. Sci. 2004, 44, 170-178; J. Med. Chem. 2004, 47, 2743-2749; J. Chem. Inf. Model. 2010, 50, 742-754; as well as SciTegic Pipeline Pilot Chemistry Collection: Basic Chemistry User Guide, March 2008, SciTegic Pipeline Pilot Data Modeling Collection, 2008-both from Accelrys Software Inc., San Diego, US, and the guides http://www.tripos.com/tripos_resources/fileroot/pdfs/Unity_111408.pdf, and http://www.tripos.com/data/SYBYL/SYBYL_072505.pdf.

An example of a similarity calculation is inserted herein below, in which a known entire side chain of a known GLP-1 derivative was compared with a methyl ester thereof:

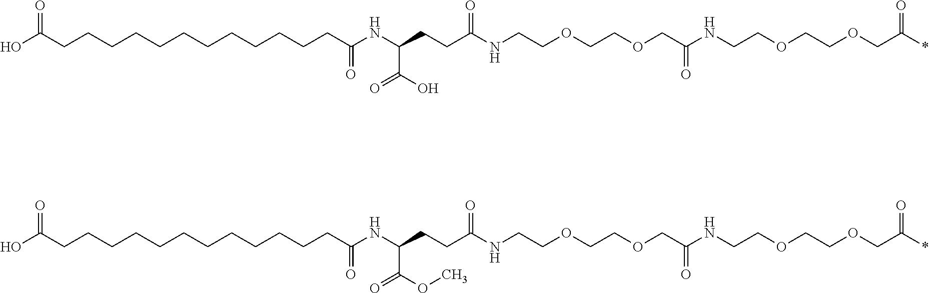 [SMILES: *C(=O)COCCOCCNC(=O)COCCOCCNC(=O)CC[C@H](NC(=O)CCCCCCCCCCCCC(=O)O)C(=O)O ; *C(=O)COCCOCCNC(=O)COCCOCCNC(=O)CC[C@H](NC(=O)CCCCCCCCCCCCC(=O)O)C(=O)OC]

Using a) ECFP_6 fingerprints the similarity is 0.798, using b) UNITY fingerprints the similarity is 0.957; and using MDL fingerprints the similarity is 0.905.

In case of two identical side chains (albumin binding moieties) the derivative may be designated symmetrical.

In particular embodiments, the similarity coefficient is at least 0.80, preferably at least 0.85, more preferably at least 0.90, even more preferably at least 0.95, or most preferably at least 0.99.

In one embodiment the GLP-1 derivative comprises a GLP-1 analogue, wherein the GLP-1 analogue comprises a a first K residue and a second K residue selected from the group consisting of i) a first K residue at a position corresponding to position 26 of GLP-1(7-37) (SEQ ID NO: 1) and a second K residue at a position corresponding to position 37 of GLP-1(7-37); and ii) a first K residue at a position corresponding to position 27 of GLP-1(7-37) (SEQ ID NO: 1) and a second K residue at a position corresponding to position T of GLP-1(7-37), where T is an integer in the range of 7-37 except 18 and 27; wherein the first K residue is designated K^{F}, and the second K residue is designated K^{T};

wherein the GLP-1 analogue comprises a maximum of ten amino acid changes as compared to GLP-1(7-37);

wherein the GLP-1 derivative comprises a first and a second protracting moiety attached to K^{F} and K^{T}, respectively, via a first and a second linker, respectively, wherein

the first and the second protracting moiety is selected from Chem. C1 and Chem. C2:

HOOC—(CH₂)ₓ—CO—* Chem. C1:

HOOC—C₆H₄—O—(CH₂)_{y}—CO—* Chem. C2:

in which x is an integer in the range of 6-16, y is an integer in the range of 3-17; and the first and second linker comprises Chem. D5:

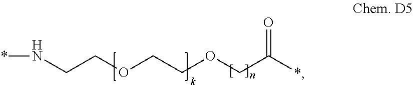

wherein k is an integer in the range of 1-5, and n is an integer in the range of 1-5; or a pharmaceutically acceptable salt, amide, or ester thereof.

In one embodiment (K^{F},K^{T}) are at positions corresponding to positions (26,37) of GLP-1(7-37) (SEQ ID NO: 1). In one embodiment (K^{F},K^{T}) are at positions corresponding to positions (27,36) of GLP-1(7-37) (SEQ ID NO: 1).

In one embodiment the GLP-1 derivative comprises the protracting moiety Chem. C2. In one embodiment Chem. C2 is represented by Chem. C2a:

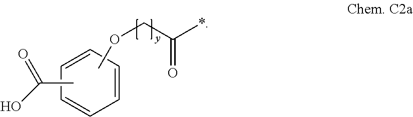

In one embodiment y of Chem. C2 or Chem. C2a is an odd number. In one embodiment y of Chem. 2 or Chem. 2a is an integer in the range of 8-11, such as 8, 9, 10 or 11. In one embodiment Chem. C2 is represented by Chem. C2b, or Chem. C2c:

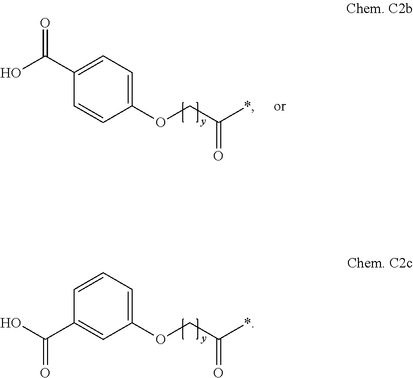

In one embodiment Chem. 5 is a first linker element. In one embodiment k of Chem. D5 is 1. In one embodiment n of Chem. D5 is 1. In one embodiment Chem. D5 is included m times, wherein m is an integer in the range of 1-10. In one embodiment m is 2. When m is not 1, then the Chem. D5 elements may be interconnected via amide bond(s).

In one embodiment the GLP-1 derivative further comprises a second linker element. In one embodiment the second linker element is a Glu di-radical. In one embodiment the second linker element is selected from Chem. E6, and/or Chem. E7:

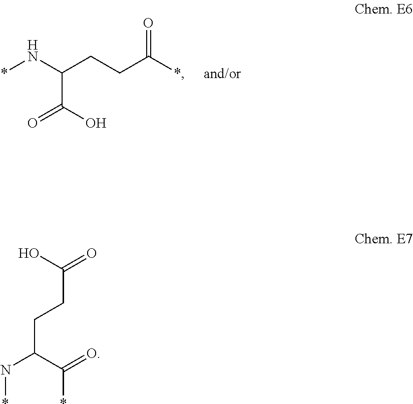

In one embodiment the second linker element is Chem. E6. In one embodiment the Glu di-radical is included p times, wherein p is an integer in the range of 1-2, such as 1 or 2. In one embodiment the second linker element comprises the Glu di-radical which is a radical of L-Glu. In one embodiment the second linker element comprises one or more Glu di-radicals and one or more Chem. D5 elements are interconnected via amide bond(s). In one embodiment the linker consists of m times Chem. D5 and p times the Glu di-radical. In one embodiment (m,p) is (2,2) or (2,1). In one embodiment (m,p) is (2,1). In one embodiment the m Chem. D5 elements and the p Glu di-radicals are interconnected via amide bonds.

In one embodiment the linker and the protracting moiety are interconnected via an amide bond. In one embodiment the linker and the GLP-1 analogue are interconnected via an amide bond. In one embodiment the linker is attached to the epsilon-amino group of the first or the second K residue.

The GLP-1 derivatives may exist in different stereoisomeric forms having the same molecular formula and sequence of bonded atoms, but differing only in the three-dimensional orientation of their atoms in space. The stereoisomerism of the exemplified derivatives of the invention is indicated in the experimental section, in the names as well as the structures, using standard nomenclature. Unless otherwise stated the invention relates to all stereoisomeric forms of the claimed derivative.

The concentration in plasma of GLP-1 derivatives may be determined using any suitable method. For example, LC-MS (Liquid Chromatography Mass Spectroscopy) may be used, or immunoassays such as RIA (Radio Immuno Assay), ELISA (Enzyme-Linked Immuno Sorbent Assay), and LOCI (Luminescence Oxygen Channeling Immunoasssay). General protocols for suitable RIA and ELISA assays are found in, e.g., WO 2009/030738 on p. 116-118.

The GLP-1 receptor agonist may be in the form of a salt, ester or amide.

In one embodiment the GLP-1 peptide is a GLP-1 derivative (a derivative of a GLP-1 analogue) acylated at 36K as well as 37K, with a side chain on eps-amino group of each of 36K and 37K, each side chain comprising a protractor of formula:

HOOC—C6H4-O—(CH2)y-CO—*, Chem. 1:

where y is an integer in the range of 8-11, attached to eps-Lys of 36K/37K, via a linker that comprises “gGlu” and/or “OEG”

*—NH—CH(COOH)—(CH2)2-CO—*,gGlu Chem. 3:

*NH—(CH2)2[O—(CH2)2]k-O—[CH2]n-CO—*, generalised Ado,

wherein k is an integer in the range of 1-5, and n is an integer in the range of 1-5;

or a pharmaceutically acceptable salt, amide, or ester thereof.

In one embodiment, the GLP-1 derivative of the present invention comprises a linker protractor peptide connections are via amide bonds at *. In another embodiment, the linker is “generalised Ado” being “OEG”, i.e. n=k=1. In another embodiment the linker is “gGlu-2xOEG”. In another embodiment, the protractor has y=10 and is in para configuration.

In some embodiments the GLP-1 receptor agonist is a GLP-1 derivative (e.g. a derivative of a GLP-1 analogue) acylated with a side chain on the epsilon-amino group of a lysine at each of positions 36 and 37;

wherein each side chain individually comprises a protractor of formula:

HOOC—C₆H₄—O—(CH₂)_{y}—CO—*, Chem. 1:

where y is an integer in the range of 8-11, attached to epsilon-amino group of a lysine at position 36 and 37; and wherein the protractor is attached to the epsilon-amino group via a linker comprising

i) gGlu of the formula:

*—NH—CH(COOH)—(CH₂)₂—CO—*, Chem. 3:

and

ii) a moiety of the formula:

*NH—(CH₂)₂—[O—(CH₂)₂]ₖ—O—[CH₂]ₙ—CO—*, Chem. 5:

wherein k is an integer in the range of 1-5, and n is an integer in the range of 1-5;

or a pharmaceutically acceptable salt, amide, or ester thereof.

In some embodiments, in the GLP-1 derivative of the invention, the linker, protractor, and peptide are connected via amide bonds at *. In some embodiments gGlu of the linker is connected to the protractor via amide bonds at *. In some embodiments gGlu of the linker is connected to the moiety of Chem. 5 via amide bonds at *. In some embodiments the moiety of Chem. 5 of the linker is connected to the peptide via amide bonds at *. In some embodiments the moiety of the formula defined by Chem. 5 is “OEG”, i.e. n=k=1. In some embodiments the linker is “*-gGlu-OEG-OEG-**” connected to the protractor at * and connected to the peptide at **. In some embodiments the protractor has y=10 and is in para configuration. In some embodiments the protractor has y=9 and is in para configuration. In some embodiments the protractor has y=9 or y=10 and is in meta configuration.

In one embodiment, the GLP-1 derivative comprises Formula II (SEQ ID 7):

Xaa7-Xaa8-Glu-Gly-Thr-Phe-Thr-Ser-Asp-Xaa16-Ser-Xaa18-Xaa19-Xaa20-Glu-Xaa22-Xaa23-Ala-Xaa25-Xaa26-Xaa27-Phe-Ile-Xaa30-Xaa31-Leu-Xaa33-Xaa34-Xaa35-Lys36-Lys37, wherein Formula II:
Xaa7 is L-histidine, (S)-2-Hydroxy-3-(1H-imidazol-4-yl)-propionic acid, D-histidine, desamino-histidine (desH), Nα-acetyl-histidine, Nα-formyl-histidine;
Xaa8 is Ala, Ser, Aib, (1-aminocyclopropyl) carboxylic acid, (1-aminocyclobutyl) carboxylic acid;
Xaa16 is Val or Leu;
Xaa18 is Ser or Arg;
Xaa19 is Tyr or Gln;
Xaa20 is Leu or Met;
Xaa22 is Gly or Glu;
Xaa23 is Gln, Glu, or Arg;
Xaa25 is Ala or Val;
Xaa26 is Arg or Lys;
Xaa27 is Glu or Leu;
Xaa30 is Ala, or Glu;
Xaa31 is Trp or His;
Xaa33 is Val or Arg;
Xaa34 is Arg, Lys, His, Asn, or Gln; and
Xaa35 is Gly or Aib.

In one embodiment the GLP-1 derivative is a GLP-1 derivative of Formula II (SEQ ID 7), wherein Xaa7 is His; Xaa8 is Aib; Xaa16 is Val; Xaa18 is Ser; Xaa19 is Tyr; Xaa20 is Leu; Xaa22 is Gly or Glu; Xaa23 is Gln; Xaa25 is Ala; Xaa26 is Arg; Xaa27 is Glu; Xaa30 is Ala or Glu; Xaa31 is Trp; Xaa33 is Val; Xaa34 is Arg or Gln; and Xaa35 is Gly.

In one embodiment the GLP-1 derivative is a GLP-1 derivative of Formula II (SEQ ID 7), wherein Xaa7 is His; Xaa8 is Aib; Xaa16 is Val; Xaa18 is Ser; Xaa19 is Tyr; Xaa20 is Leu; Xaa22 is Glu; Xaa23 is Gln; Xaa25 is Ala; Xaa26 is Arg; Xaa27 is Glu; Xaa30 is Ala; Xaa31 is Trp; Xaa33 is Val; Xaa34 is Arg; and Xaa35 is Gly.

### Exenatide

Exenatide is a commercial incretin mimetic for the treatment of diabetes mellitus type 2, which is manufactured and marketed by Amylin Pharmaceuticals and Eli Lilly & Co. Exenatide is based on Exendin-4 or Exendin-3, a hormone found in the saliva of the Gila monster (Heloderma suspectum and Heloderma horridum), that displays biological properties similar to human GLP-1. U.S. Pat. No. 5,424,286 relates i.e. to a method of stimulating insulin release in a mammal by administration of Exendin-4(1-39) (SEQ ID 3).

The Gila monster Exendin-4(1-39) sequence is:

(SEQ ID 3)
HGEGTFTSDL SKQMEEEAVR LFIEWLKNGG PSSGAPPPS,

while the sequence of Exendin-4(1-29) is:

(SEQ ID 5)
HGEGTFTSDL SKQMEEEAVR LFIEWLKNG

while the sequence of Exendin-3(1-39) is:

Exendin-3(1-39):
(SEQ ID 2)
HSDGTFTSDLSKQMEEEAVRLFI EWLKNGGPSSGAPPPS.

For the purposes of numbering in Formula I (SEQ ID 4), the start position X₇ of Formula I corresponds to histidine in position 1 of Exendin-4 or Exendin-3 (SEQ ID Nos. 2, 3 and 5), and ends in position X₃₇, corresponding to position 31 in Exendin-4 or Exendin-3 sequence (SEQ ID Nos. 2 and 3), or position X₄₅, corresponding to position 39 in Exendin-4 (SEQ ID 2 and 3).

However, as for the sequence listing, the first amino acid residue of SEQ ID Nos. 2, 3 and 5 (histidine or X₇ of Formula I) is assigned No. 1. Exendin-4 or Exendin-3 amino acids positions 1 to 39 in SEQ ID Nos. 2 and 3 are to be the same as amino acid positions X₇ to X₄₅ of Formula I (SEQ ID 4). Likewise, amino acid positions 1 to 29 of Exendin-4 (1-29) (SEQ ID 5) are to be the same as amino acid positions X₇ to X₃₅. For the purposes of numbering in Formula I (SEQ ID 4), the first amino acid residue (histidine) of SEQ ID Nos. 2, 3 and 4 is assigned X₇.

### Preparation of GLP-1 Peptides

The production of polypeptides and peptides such as GLP-1 peptide is well known in the art. The GLP-1 peptides for use in the invention may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The GLP-1 peptides may also be produced by a method which comprises culturing a host cell containing a DNA sequence encoding the GLP-1 peptide and capable of expressing the GLP-1 peptide in a suitable nutrient medium under conditions permitting the expression of the peptide. For GLP-1 peptides comprising non-natural amino acid residues, the recombinant cell should be modified such that the non-natural amino acids are incorporated into the GLP-1 peptide, for instance by use of tRNA mutants.

In one embodiment a tablet core according to the present invention comprises a GLP-1 peptide as disclosed and claimed in patent applications WO2006/097537, WO2011/080103 and WO2012/140117. A non-limiting list of examples of GLP-1 peptides according to the invention may e.g. be found in WO 2006/097537, WO 2011/080103 and WO2012/140117. Methods for preparation of GLP-1 peptides of the present invention can for example be found in WO2006/097537, WO2011/080103 and WO2012/140117. Additional methods for preparation of GLP-1 peptides of the present invention may be found in PCT/EP2015/057442. Methods for preparation of such GLP-1 peptides as well as assays for characterizing such GLP-1 peptides, such as physical and chemical stability as well as potency and T_{1/2} are provided in WO2006/097537, WO2011/080103 and WO2012/140117.

The GLP-1 receptor agonist may be semaglutide, see for example of example 1; semaglutide is also referred to as Compound A herein. Semaglutide may be prepared as disclosed e.g. in Example 4 of WO2006/097537. Semaglutide is N^{ε26}-{18-[N-(17-carboxyheptadecanoyl)-L-γ-glutamyl]-10-oxo-3,6,12,15-tetraoxa-9,18-diazaoctadecanoyl}-[8-(2-amino-2-propanoic acid),34-L-arginine]human glucagon-like peptide 1(7-37) (WHO Drug Information Vol. 24, No. 1, 2010) and has the following structure:

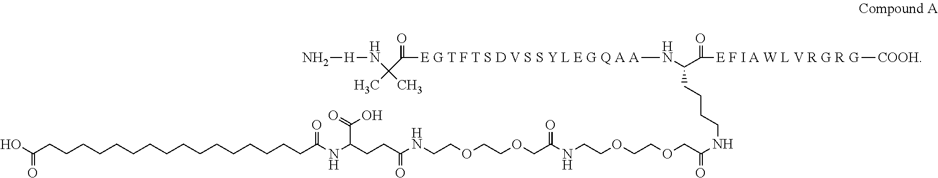

The GLP-1 receptor agonist may be Compound B, see for example example 2 herein. Compound B may be prepared as disclosed in e.g. Example 2 of WO2011/080103. Compound B is N^{ε26}{2-[2-(2-{2-[2-(2-{(S)-4-Carboxy-4-[10-(4-carboxyphenoxy)decanoylamino]butyrylamino}ethoxy) ethoxy]acetylamino}ethoxy)ethoxy]acetyl}, N^{ε37}-{2-[2-(2-{2-[2-(2-{(S)-4-carboxy-4-[10-(4-carboxyphenoxy)decanoylamino]butyrylamino}ethoxy)ethoxy] acetylamino}ethoxy)ethoxy]acetyl}-[Aib⁸,Arg³⁴,Lys³⁷]GLP-1(7-37)-peptide and has the following structure:

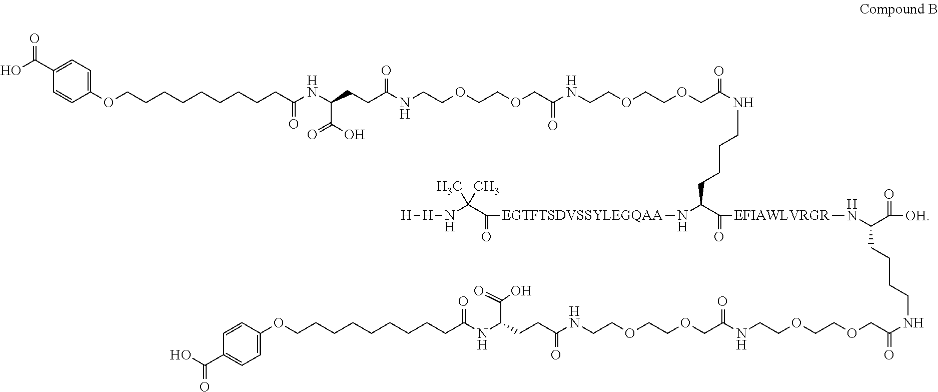

The GLP-1 receptor agonist may be Compound C, see for example example 3 herein. Compound C may be prepared as disclosed in e.g. Example 31 of WO2012/140117. Compound C is N^{ε27}-[2-[2-[2-[[2-[2-[2-[[(4S)-4-carboxy-4-[10-(4-carboxyphenoxy)decanoylamino]butanoyl]amino]ethoxy]ethoxy] acetyl]amino]ethoxy]ethoxy]acetyl], N^{ε36}-[2-[2-[2-[[2[2-[2-[[(4S)-4-carboxy-4-[10-(4-carboxyphenoxy)decanoylamino]butanoyl]amino]ethoxy]ethoxy]acetyl]amino]ethoxy] ethoxy]acetyl]-[Aib8,Glu22,Arg26,Lys27,Glu30,Arg34,Lys36]-GLP-1-(7-37)-peptidyl-Glu-Gly and has the following structure:

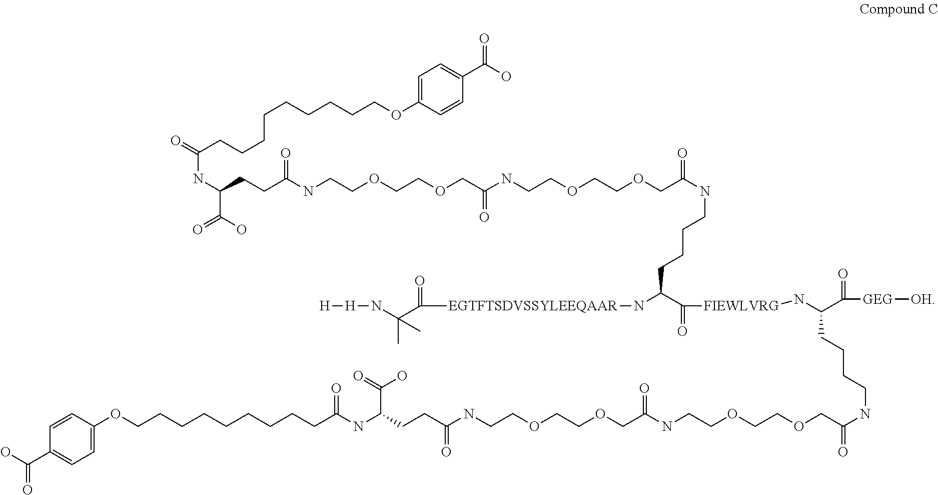

The GLP-1 receptor agonist may be Compound D. Compound D may be prepared as disclosed in e.g. WO2012/140117 or Example 1 of PCT/EP2015/057442. Compound D is N{Epsilon-36}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl]-[Aib8,Glu22,Arg26,Arg34,Lys36,Lys37]-GLP-1-(7-37)-peptide

and has the following structure:

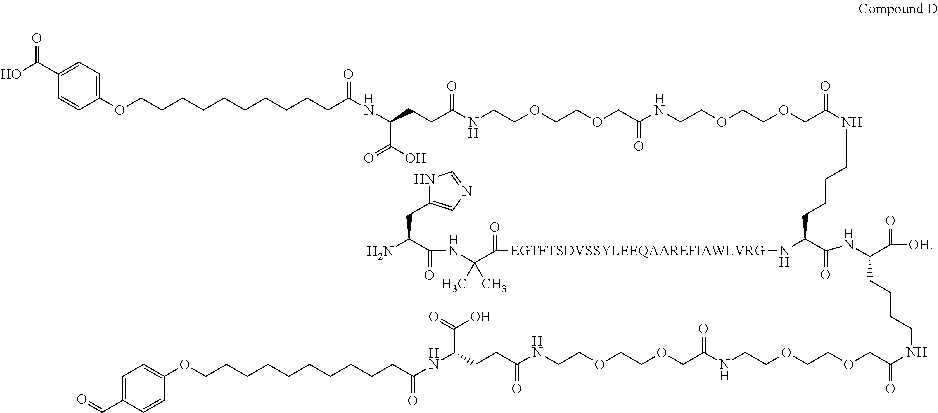

The GLP-1 receptor agonist may be Compound E. Compound E may be prepared as disclosed in e.g. WO2012/140117 or Example 2 of PCT/EP2015/057442. Compound E is N{Epsilon-36}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl]-[Aib8,Arg26,Arg34,Lys36,Lys37]-GLP-1-(7-37)-peptide

and has the following structure:

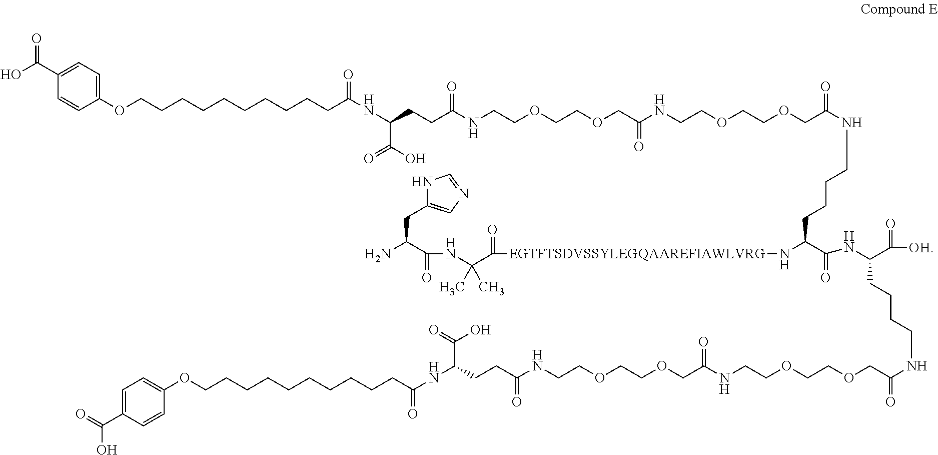

In some embodiments Compound D and Compound E may be prepared according to methods known by a person skilled in the art.

The GLP-1 receptor agonist may be Compound F. Compound F may be prepared as disclosed in e.g. WO2012/140117 or Example 35 of PCT/EP2015/057442. In one embodiment, the GLP-1 peptide is N{Epsilon-36}-[2-[2-[2-[[2-[2-[2-[[(4S)-4-carboxy-4-[10-(3-carboxyphenoxy)decanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[10-(3-carboxyphenoxy)decanoylamino]butanoyl]amino]ethoxy]ethoxy]acetyl]amino]ethoxy]ethoxy]acetyl]-[Aib8,Glu22,Arg26,Arg34,Lys36,Lys37]-GLP-1-(7-37)-peptide

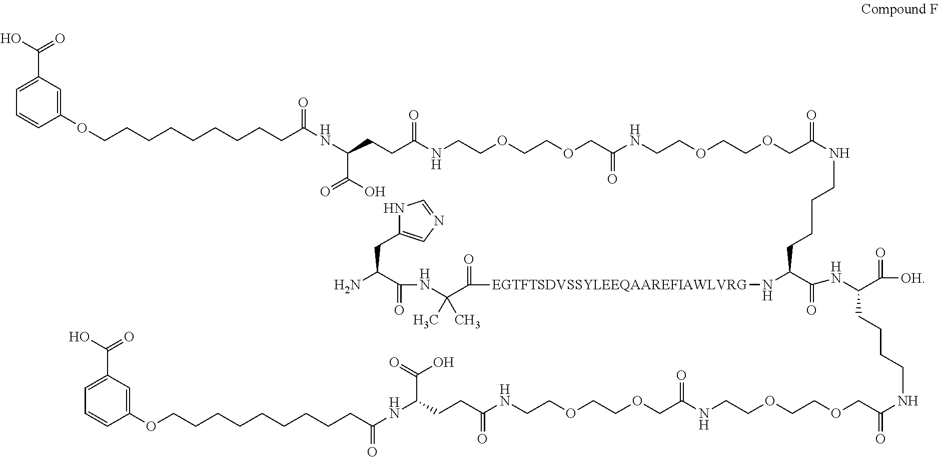

### Further Excipients of the Core

In one embodiment a composition according to the present invention comprises a tablet core, wherein said tablet core comprises one or more excipients, such as one or more polyols and/or one or more lubricants. In one embodiment a composition according to the present invention comprises one or more polyols. In one embodiment a composition according to the present invention comprises a tablet core, wherein said tablet core comprises one or more polyols, such as, but not limited to one or more of sorbitol and mannitol. In one embodiment a composition according to the present invention comprises one or more polyols, wherein said polyols are selected from the group consisting of sorbitol, mannitol and mixtures thereof.

In one embodiment a composition according to the present invention comprises a tablet core, wherein said tablet core comprises lubricants, such as, but not limited to stearic acid, magnesium stearate, stearate and colloidal silica. In one embodiment a composition according to the present invention comprises lubricants, wherein said lubricants are selected from the group consisting of stearic acid, magnesium stearate, stearate or mixtures thereof.

In one embodiment a composition according to the present invention comprises a tablet core, wherein said tablet core comprises an absorption enhancer and one or more excipients. In one embodiment, said one or more excipients further comprised in the tablet core is selected from the group consisting of sorbitol, magnesium stearate, stearate and stearic acid.

In one embodiment a tablet core comprises about 0.5% (w/w) stearic acid.

In one embodiment a tablet core comprises about 22% (w/w) sorbitol.

In one embodiment the sorbitol amount is adjusted relative to the amount of active ingredient such as e.g. the GLP-1 peptide. In one embodiment the sorbitol amount is adjusted relative to the amount of GLP-1 peptide. In one embodiment the sorbitol amount is adjusted relative to the amount of GLP-1 peptide after the principle of quantum satis (QS) meaning the amount which is needed to obtain a tablet with the desired weight.

In certain embodiments of the present invention, the pharmaceutical composition comprises a tablet core, wherein said tablet core may comprise additional excipients commonly found in a pharmaceutical composition, examples of such excipients include, but are not limited to fillers, disintegrants, enzyme inhibitors, stabilizers, preservatives, flavors, sweeteners and other components as described in ‘Handbook of Pharmaceutical Excipients’ Ainley Wade, Paul J. Weller, Arthur H. Kibbe, 3^{rd} edition, American Pharmacists Association (2000) or—‘Handbook of Pharmaceutical Excipients’, Rowe et al., Eds., 4th Edition, Pharmaceutical Press (2003).

### Pharmaceutical Composition

In one embodiment the pharmaceutical composition of the present invention, comprises more than one core (e.g. mini-tablets, optionally comprised in larger unit, such as a tablet or a capsule) and one coating (e.g. located on the surface of each core and/or on the surface of the larger unit).

In one embodiment a tablet core of a composition according to the present invention is a mini-tablet, a tablet or a capsule. In one embodiment a tablet core of a composition according to the present invention weighs between 3.0 and 800 mg. In one embodiment a tablet core of a composition according to the present invention is a tablet weighing up to 800 mg. In one embodiment a tablet core of a composition according to the present invention is a capsule weighing up to 1000 mg.

In one embodiment a tablet core of a composition according to the present invention weighs about 710 mg. In one embodiment a composition according to the present invention consisting of a tablet core and an immediate release coating weighs about 728 mg.

The compositions of the present invention may be in the form of a tablet. In some embodiments the weight of the tablet is in the range of 175 mg to 1000 mg, such as in the range of 175-250 mg, 300-500 mg or 500-900 mg, or such as about 200 mg, about 400 mg or about 700 mg. In some embodiments the weight of the tablet is in the range of 200 mg to 1000 mg, such as in the range of 500-700 mg or 600-1000 mg, or such as about 200 mg, about 400 mg, about 600 mg or about 800 mg.

The compositions of the present invention may be in the form of a mini-tablet. In some embodiments the weight of the mini-tablet is in the range of 3 mg to 200 mg, such as in the range of 5-15 mg, 15-50 mg, 50-150 mg, or 120-160, such as about 8 mg, about 12 mg, about 30, about 40 mg or about 140 mg.

In one embodiment a tablet core of a composition according to the present invention is a mini-tablet weighing up to 175 mg.

In one embodiment a mini-tablet core of the composition according to the present invention weighs about 4 mg. In one embodiment a composition according to the present invention consisting of a mini-tablet core and an immediate release coating weighs about 4.3 mg.

In one embodiment a tablet core of a composition according to the present invention is a multiparticulate system. In one embodiment a tablet core of a composition according to the present invention is a multiparticulate system, wherein said multiparticulate system may be compressed into the form of a tablet or contained in a capsule. The multiparticulate system can be in the form of a tablet or capsule.

In one embodiment a capsule according to the present invention comprises up to 100 mini-tablets. In one embodiment a capsule according to the present invention comprises up to 150 mini-tablets. In one embodiment a capsule according to the present invention comprises up to 200 mini-tablets. In one embodiment a capsule according to the present invention comprises up to 300 mini-tablets. In one embodiment a capsule according to the present invention comprises about 178 mini-tablets.

In one embodiment a tablet core according to the present invention comprises one or more layers. The tablet can be a single or multilayer tablet having a compressed multiparticulate system in one, all or none of the layers.

In one embodiment a tablet core according to the present invention comprises one or more tablets. In one embodiment a tablet core according to the present invention comprises up to three tablets. In one embodiment a tablet core according to the present invention comprises two tablets. In one embodiment a tablet core according to the present invention comprises two or more tablets.

In one embodiment a tablet core according to the present invention is a multiparticulate system comprising particles of the same dimensions. In one embodiment a tablet core according to the present invention is a multiparticulate system comprising particles of various dimensions.

In one embodiment particles of multiparticulate systems according to the present invention are coated with an immediate release coating. In one embodiment particles of multiparticulate systems according to the present invention are coated with an immediate release coating, wherein said immediate release coating is an Opadry®II coating.

In one embodiment particles of multiparticulate systems according to the present invention are individually coated with an immediate release coating. In one embodiment particles of multiparticulate systems according to the present invention are individually coated with an immediate release coating, before pressed into a tablet.

In one embodiment individually coated particles of a multiparticulate system according to the present invention are pressed into a tablet core. In one embodiment individually coated particles of a multiparticulate system according to the present invention are pressed into a tablet core and the resulting tablet core is not coated with another layer of immediate release coating. In one embodiment individually coated particles of a multiparticulate system according to the present invention are pressed into a tablet core and said resulting tablet core is also coated with an immediate release coating. In one embodiment particles of multiparticulate systems according to the present invention are individually coated with immediate release coating and pressed into a tablet and said resulting tablet is coated with an additional immediate release coating.

In one embodiment particles of multiparticulate systems according to the present invention are collectively coated with an immediate release coating. In one embodiment particles of multiparticulate systems according to the present invention are collectively coated with an immediate release coating, after being pressed into a tablet.

In one embodiment of the present invention relates to a pharmaceutical composition comprising a tablet core and an immediate release coating, wherein said immediate release coating according to the present invention dissolves in aqueous medium at any pH.

In one embodiment, the pharmaceutical composition completely disintegrates into the medium within between 5-10 minutes, such as between 6-9 minutes, wherein the disintegration test is carried out as defined in the European Pharmacopeia in type 1 water, such as e.g. milli-Q water, at 37° C. The term “completely disintegrates” is herein a measure of the state wherein no residue of the pharmaceutical composition remains on the screen of the test apparatus. In one embodiment, a pharmaceutical composition of the invention is completely disintegrated when it is disintegrated into particles that are less than 50 μm in diameter, i.e. particles that will pass through a 50 mesh screen.

In one embodiment, the pharmaceutical composition does not comprise a protease inhibitor, such as a Bowman-Birk inhibitor.

### Indications

The present invention also relates to a pharmaceutical composition of the invention, for use as a medicament.

In particular embodiments, the pharmaceutical composition of the invention may be used for the following medical treatments:

(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;

(ii) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;

(iii) improving β-cell function, such as decreasing β-cell apoptosis, increasing β-cell function and/or β-cell mass, and/or for restoring glucose sensitivity to β-cells;

(iv) prevention and/or treatment of cognitive disorders and/or neurodegenerative disorders, such as Alzheimer's disease, Parkinson's disease, and/or multiple sclerosis;

(v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;

(vi) prevention and/or treatment of diabetic complications, such as angiopathy; neuropathy, including peripheral neuropathy; nephropathy; and/or retinopathy;

(vii) improving lipid parameters, such as prevention and/or treatment of dyslipidemia, lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein a (apo(a)) in vitro and/or in vivo;

(viii) prevention and/or treatment of cardiovascular diseases, such as syndrome X, atherosclerosis, myocardial infarction, coronary heart disease, reperfusion injury, stroke, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atheroschlerosis oblitterens), diastolic dysfunction, and/or systolic dysfunction; and/or reduction of blood pressure, such as reduction of systolic blood pressure;

(ix) prevention and/or treatment of gastrointestinal diseases, such as inflammatory bowel disease, short bowel syndrome, or Crohn's disease or colitis; dyspepsia, and/or gastric ulcers; and/or inflammation, such as psoriasis, psoriactic arthritis, rheumatoid arthritis, and/or systemic lupus erythematosus;

(x) prevention and/or treatment of critical illness, such as treatment of a critically ill patient, a critical illness poly-nephropathy (CIPNP) patient, and/or a potential CIPNP patient; prevention of development of critical illness or CIPNP; prevention, treatment and/or cure of systemic inflammatory response syndrome (SIRS) in a patient; prevention or reduction of the likelihood of a patient suffering from bacteraemia, septicaemia, and/or septic shock during hospitalisation; and/or stabilising blood glucose, insulin balance and optionally metabolism in intensive care unit patients with acute illness;

(xi) prevention and/or treatment of polycystic ovary syndrome (PCOS);

(xii) prevention and/or treatment of cerebral disease, such as cerebral ischemia, cerebral haemorrhage, and/or traumatic brain injury;

(xiii) prevention and/or treatment of sleep apnoea; and/or

(xiv) prevention and/or treatment of abuse, such as alcohol abuse and/or drug abuse.

In a particular embodiment the indication is selected from the group consisting of (i)-(xiv), such as indications (i)-(viii), (x)-(xiii), and/or (xiv), and relates in one way or the other to diabetes.

In another particular embodiment, the indication is selected from the group consisting of (i)-(iii) and (v)-(viii), such as indications (i), (ii), and/or (iii); or indication (v), indication (vi), indication (vii), and/or indication (viii).

In a still further particular embodiment, the indication is (i). In a further particular embodiment the indication is (v). In a still further particular embodiment the indication is (viii).

The following indications are particularly preferred: Type 2 diabetes, and/or obesity.

The invention may also solve further problems that will be apparent from the disclosure of the exemplary embodiments.

In one embodiment a composition according to the present invention shows a Tmax which is less than 3 hours after oral administration to a Beagle dog.

In one embodiment a composition according to the present invention is in the form of a solid oral composition. In one embodiment a composition according to the present invention is manufactured into a tablet. In one embodiment a composition according to the present invention is manufactured into a tablet for oral administration.

### Methods for Producing

In one embodiment of the present invention provides a method for manufacture of compositions according to the present invention.

### Coating

In one embodiment the immediate release coating of the present inventions is performed by any methods known to the person skilled in the art. In one embodiment the coating of the present invention is performed by any method disclosed in “Coating processes and equipment, by D. M. Jones in “Pharmaceutical dosage forms: Tablets”, Informa Healthcare, N.Y., vol 1, 2008 p 373-399, L. L. Augsburger and S. W. Hoag”.

In one embodiment the immediate release coating dispersion is filtrated through a mesh filter prior to the actual coating prior to the actual coating procedure. In one embodiment the immediate release coating dispersion is allowed to be stirred prior to a filtration through a mesh filter, prior to the actual coating procedure. In one embodiment the immediate release coating dispersion is allowed to be stirred prior to a filtration through an about 0.24 mm mesh filter, prior to the actual coating procedure.

In one embodiment the immediate release coating dispersion comprising further excipients is filtrated through a mesh filter prior to the actual coating prior to the actual coating procedure. In one embodiment the immediate release coating dispersion comprising further excipients is allowed to be stirred prior to a filtration through a mesh filter, prior to the actual coating procedure. In one embodiment the immediate release coating dispersion comprising further excipients is allowed to be stirred prior to a filtration through an about 0.24 mm mesh filter, prior to the actual coating procedure.

In one embodiment the actual coating procedure of tablet cores or tablets according to the present invention is performed in a pan coater or fluid bed coater. In one embodiment the actual coating procedure of tablet cores or tablets according to the present invention is performed in a pan coater or fluid bed coater by spraying the immediate release coating dispersion through a spray nozzle. In one embodiment the actual coating procedure of tablet cores or tablets according to the present invention is performed in a pan coater or fluid bed coater by spraying the immediate release coating dispersion further comprising further excipients through a spray nozzle.

In one embodiment said coating processes and equipment may be used as disclosed by D. M. Jones in “Pharmaceutical dosage forms: Tablets”, Informa Healthcare, N.Y., vol. 1, 2008 p 373-399, L. L. Augsburger and S. W. Hoag”.

### Tablet Core

In one embodiment the tablet core is manufactured by suitable methods for formulation of oral compositions.

In one embodiment a GLP-1 receptor agonist powder is sieved before formulation. In one embodiment a sorbitol (or a polyol or any other equivalent excipient) powder is sieved before formulation. In one embodiment sorbitol and GLP-1 receptor agonist powder are mixed together. In one embodiment equal amounts of sorbitol and GLP-1 receptor agonist powder are mixed by hand.

In one embodiment sorbitol and GLP-1 receptor agonist powders are mixed by hand and by an automatized mixing process. In one embodiment sorbitol and GLP-1 receptor agonist powders are mixed by hand and by an automatized mixing process, wherein said automatized mixing process is performed in a Tubular-mixer.

In one embodiment sorbitol and GLP-1 receptor agonist powders are initially mixed by an automatized mixing process. In one embodiment equal amounts of sorbitol and GLP-1 receptor agonist powder are mixed by hand and another portion of sorbitol is added in an amount twice as high as the first addition of sorbitol, which then is also stirred well by hand. When said last addition of sorbitol is admixed well, the powder is then subjected to mechanical mixing in a Turbula-mixer or any equivalent mixer to finalise the mixing process, resulting in a homogenous powder.

In one embodiment a salt of capric acid is added to said homogenous powder of sorbitol and GLP-1 receptor agonist in amounts of 1:1. The addition may be performed in two steps and the mixing may initially performed by hand and finalised by mechanical mixing in a Turbula-mixer or any other automatized mixing device. The addition may be performed in two steps and the mixing is initially performed by hand and finalised by mechanical mixing in a Turbula-mixer or any equivalent mixer.

The powder may then be pressed in a tablet press as known to the person skilled in the art, resulting in a tablet core according to the present invention.

The powder may then be pressed in a rotary tablet press as known to the person skilled in the art, resulting in a tablet core according to the present invention. The powder may then be pressed in a single punch tablet press as known to the person skilled in the art, resulting in a tablet core according to the present invention. The powder may then be pressed in an excenter tablet press as known to the person skilled in the art, resulting in a tablet core according to the present invention.

### Bioavailability and Onset of Action

With the term “oral bioavailability” is herein meant the fraction of the administered dose of active pharmaceutical ingredient (API) that reaches the systemic circulation after having been administered orally compared to when administered intravenously. By definition, when an active pharmaceutical ingredient (API) is administered intravenously, its bioavailability is 100%.

Generally, the term bioavailability refers to the fraction of an administered dose of the active pharmaceutical ingredient (API), such as a GLP-1 peptide comprised in a pharmaceutical composition of the invention, that reaches the systemic circulation unchanged. However, when it is administered via other routes (such as orally), its bioavailability decreases (due to incomplete absorption and first-pass metabolism).

Absolute oral bioavailability compares the bioavailability (determined as the area under the curve, or AUC) of the API in systemic circulation following oral administration, with the bioavailability of the same dose of same API following intravenous administration. It is the fraction of the API absorbed through non-intravenous administration compared with the corresponding intravenous administration of the same dose of same API. The comparison must be dose normalised if different doses are used, consequently each AUC is corrected by dividing the corresponding dose administered.

A plasma API concentration versus time plot is made after both oral and intravenous administration. The absolute bioavailability (F) is the dose-corrected AUC-oral divided by dose-corrected AUC-intravenous.

Standard methods for determining bioavailability of an API such as a GLP-1 receptor agonist are known to the person skilled in the art and include inter alia measurement of the relative areas under the curve (AUC) for the concentration of the GLP-1 receptor agonist in question administered orally and intra venously (i.v.) in the same species. Quantitation of GLP-1 receptor agonist concentrations in blood (plasma) samples can be done using for example antibody assays (ELISA) or by mass spectrometry.

However, when a drug is administered orally the bioavailability of the active ingredient decreases due to incomplete absorption and first-pass metabolism. The plasma concentration of a GLP-1 receptor agonist may be measured in an assay as known by a person skilled in the art as e.g. described in WO 2011/080103 (see e.g. page 17-18). In one embodiment, the bioavailability of the pharmaceutical composition of the invention is at least 0.5% such as at least 1% or at least 2%. In another embodiment, bioavailability of the pharmaceutical composition of the invention is at least 2.3%, at least 2.6%, at least 3.0%, at least 3.3%, at least 3.6%, at least 4.0%, at least 4.1%, at least 4.2%, at least 4.3%, at least 4.4% or at least 4.5%. In another embodiment, the bioavailability of the pharmaceutical composition of the invention is between 2 and 4%.

The term “Tmax” as used herein means the time after administration of a drug when the maximum plasma concentration an API, herein GLP-1 receptor agonist, is reached (i.e. Cmax).

The term “Cmax” as used herein means the peak plasma concentration of an API, herein GLP-1 receptor agonist.

Standard methods for determining the pharmacokinetics of the GLP-1 receptor agonist of the pharmaceutical composition of the invention are known to the person skilled in the art and include inter alia measurement of the concentration of the GLP-1 receptor agonist in question administered orally and intra venously (i.v.) in the same species. Quantitation of GLP-1 receptor agonist concentrations in blood (plasma) samples can be done using for example antibody assays (ELISA) or by mass spectrometry.

In one embodiment, onset of action is less than 3 hours. Tmax may be a measure of the onset of action. In one embodiment, Tmax is less than 3 hours.

### Terms and Definitions

In the present context, if not stated otherwise, the terms “dissolves in water”, “dissolves in aqueous medium” and “is soluble in aqueous medium” may be used interchangeably and refer to the solubility of an excipient in water or in an aqueous salt or aqueous buffer solution, or in an aqueous solution containing other compounds.

As used herein the term “dissolves at all pH values” means it dissolves in aqueous medium and/or dissolves throughout the entire pH range and at any pH in aqueous medium.

The term “enteric coating” as used herein means a polymer coating that controls disintegration and release of the solid oral dosage form. The site of disintegration and release of the solid dosage form may be customized depending on the enteric coating ability to resist disintegration in a specific pH range.

The term “onset of action” as used herein means the time after administration of a drug it takes to attain a pharmacological relevant plasma concentration.

The terms “medium-chain fatty acid” and “medium-chain fatty diacid” are herein used for fatty acids respectively fatty diacids having a medium length carbon chain such as e.g. carbon chains with between 6 to 12 carbon atoms. Non limiting examples of medium-chain fatty acids and diacids include hexanoic acid, octanoic acid, decanoic acid, dodecanoic acid, hexanedioic acid, octanedioic acid, decanedioic acid and dodecanedioic acid.

The term “immediate release coating dispersion” as used herein includes solutions and dispersion, i.e. situations where the immediate release coating is partly or completely dissolved in said aqueous medium. In one embodiment a dispersion of water and said immediate release coating material is placed in a beaker on a suitable stirring apparatus.

The terms “disintegration”, “disintegrating”, “disintegrate” or “disintegrated” as used herein and when referring to a pharmaceutical composition comprising a tablet core and an immediate release coating, is to be understood as said pharmaceutical composition being disintegrated into components, wherein some or all of the components are completely dissolved into the medium triggering said disintegration. Disintegration may be measured according to methods understood by the person skilled in the art. I one embodiment disintegration is determined as defined in the European Pharmacopeia.

The terms “dissolution”, “dissolving”, “dissolve” or “dissolved” as used herein and when referring to a coating, is to be understood as said coating being completely dissolved into the medium triggering said dissolution. Dissolution may be measured according to methods understood by the person skilled in the art. Dissolution of an immediate release coating may e.g. be determined as defined in the European Pharmacopeia such as e.g. by paddle or basket dissolution method.

Herein, the term “dispersion” means a dispersion, an emulsion or a system consisting of two non-miscible components.

The term “about” as used herein means in reasonable vicinity of the stated numerical value, such as plus or minus 10%. The terms “mainly” and “majority” as used herein is a quantification to indicate; a part, area, size and frequency that is greater than 50% including about 60%, 70%, 80%, 90% or more relative to the context that it refers to.

The term “stability” is herein used for a pharmaceutical composition comprising GLP-1 receptor agonist to describe the shelf life of the composition.

As used herein, “stabilized” or “stable” when referring to a pharmaceutical composition of the invention refers to a pharmaceutical composition with increased chemical stability, increased physical stability or increased chemical and physical stability relative to a pharmaceutical composition without all the ingredients of the pharmaceutical composition of the present invention. In general, a pharmaceutical composition must be stable during use and storage (in compliance with recommended use and storage conditions) until the expiration date is reached.

The term “immediate release coating” is known to the person skilled in the art. Thus, as used herein, this term discloses coatings that are released immediately when contacted with any solution, being pH independent. The term also includes what the skilled person in the art appreciates as an “immediate release coating film”. Coatings for immediate release tablets are typically comprised of a polymer, a plasticizer and optionally anti-tacking and/or pigment/opacifier. In one embodiment, an immediate release coating according to invention is a coating or film coating which comprises one or more types of immediate release coating polymers such as e.g. one or more polyvinyl alcohol polymers and/or HPMC polymers.

Herein the term “film coating” or “film coat” is used for a thin layer of polymeric material (i.e. immediate release coating) surrounding a tablet, capsule, or mini-tablet. In one embodiment a film coating has a layer of up to 8 mg immediate release coating per cm² tablet surface. In one embodiment a film coating has a layer of up to 4 mg immediate release coating per cm² tablet surface.

The term “coating based on immediate release coating polymer” as used herein refers to a coating which comprises immediate release coating polymers and/or copolymers, for example, comprises 20% (w/w) or more immediate release coating polymers and/or copolymers. A coating based on immediate release coating polymer is comprised by the term “immediate release coating”.

The term “immediate release coating material” as used herein refers to the material which is purchased or produced, often a dry powder, and comprises all components of the immediate release coating.

The terms “polyvinyl alcohol based coating” and “hydroxypropylmethyl-cellulose based coating” are herein used for coatings based on polyvinyl alcohol (PVA) polymer respectively hydroxypropylmethylcellulose (alternatively named hypromellose or HPMC) polymer and include without limitation coatings based on either PVA or HPMC as the only polymer, PVA or HPMC used together with other polymers and PVA or HPMC cross-linked to other polymers such as e.g. polyethylene glycol (PEG). For example, a coating comprising e.g. PVA crosslinked to PEG is comprised by the term polyvinyl alcohol based coating. The PVA and/or HPMC based coating may comprise further excipients, such as for example, but not limited to, a plasticizer and a pigment.

The term “GLP-1 receptor agonist powder” as used herein refers to the active pharmaceutical ingredient (API), which is stored in the form of a powder, in this case the API is an acylated GLP-1 peptide as herein defined, therefore the powder is a “GLP-1 receptor agonist powder”.

The term “sorbitol powder” as used herein refers to any sorbitol or equivalent excipient, such as mannitol, which is stored in the form of a powder.

As use herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

### FURTHER EMBODIMENTS OF THE INVENTION

1. A pharmaceutical composition comprising a tablet core and an immediate release coating, wherein said tablet core comprises an absorption enhancer and a GLP-1 receptor agonist.
2. The pharmaceutical composition according to embodiment 1, wherein said immediate release coating dissolves in aqueous medium at any pH.
3. The pharmaceutical composition according to any one of the previous embodiments, wherein said immediate release coating is selected from a polyvinyl alcohol based coating and a hydroxypropylmethylcellulose based coating.
4. The pharmaceutical composition according to any one of the previous embodiments, wherein said immediate release coating comprises polyvinyl alcohol.
5. The pharmaceutical composition according to any one of the previous embodiments, to the extent possible, wherein said immediate release coating comprises about 25-55% polyvinyl alcohol.
6. The pharmaceutical composition according to any one of the preceding embodiments, to the extent possible, wherein said immediate release coating comprises about 38-46% polyvinyl alcohol.
7. The pharmaceutical composition according to any one of the previous embodiments, to the extent possible, wherein said immediate release coating is selected from Opadry®, Opadry®Clear, Opadry®II, Opadry®II Clear, Opadry®II Pigmented or Opadry®II Yellow.
8. The pharmaceutical composition according to any one of the preceding embodiments, to the extent possible, wherein said immediate release coating is Opadry®II.
9. The pharmaceutical composition according to any one of the preceding embodiments, to the extent possible, wherein said immediate release coating is Opadry®.
10. The pharmaceutical composition according to any one of the preceding embodiments, to the extent possible, wherein said immediate release coating is Opadry® clear.
11. The pharmaceutical composition according to any one of the preceding embodiments, to the extent possible, wherein said immediate release coating is selected from Opadry®II clear or Opadry®II Pigmented.
12. The pharmaceutical composition according to any one of the preceding embodiments, to the extent possible, wherein said immediate release coating is Opadry®II clear.
13. The pharmaceutical composition according to any one of the preceding embodiments, to the extent possible, wherein said immediate release coating is Opadry®II Pigmented.
14. The pharmaceutical composition according to any one of the preceding embodiments, to the extent possible, wherein said immediate release coating is Opadry®II Yellow.
15. The pharmaceutical composition according to any one of the preceding embodiments, to the extent possible, wherein said immediate release coating is Opadry®II Yellow is in the amount of 4.4 to 8.8 mg/cm2.
16. The pharmaceutical composition according to any one of the preceding embodiments, to the extent possible, wherein said immediate release coating is Opadry®II Yellow is in the amount of about 4.4 mg/cm2.
17. The pharmaceutical composition according to any one of the preceding embodiments, to the extent possible, wherein said immediate release coating is Opadry®II Yellow is in the amount of about 8.8 mg/cm2.
18. The pharmaceutical composition according to any one of the preceding embodiments, to the extent possible, wherein said immediate release coating is Opadry® clear is in the amount of 3.9 to 4.4 mg/cm2.
19. The pharmaceutical composition according to any one of the preceding embodiments, to the extent possible, wherein said immediate release coating is Opadry® clear is in the amount of about 4.4 mg/cm2.
20. The pharmaceutical composition according to any one of the preceding embodiments, wherein said absorption enhancer is a salt of a medium-chain fatty acid.
21. The pharmaceutical composition according to any one of the preceding embodiments, wherein said medium-chain fatty acid is capric acid.
22. The pharmaceutical composition according to any one of the preceding embodiments, wherein said medium-chain fatty acid is a salt of capric acid.
23. The pharmaceutical composition according to any one of the preceding embodiments, wherein said salt of a medium-chain fatty acid is sodium caprate.
24. The pharmaceutical composition according to any of the preceding embodiments, wherein said tablet core further comprises one or more excipients.
25. The pharmaceutical composition according to any of the preceding embodiments, said further excipients are sorbitol and stearic acid.
26. The pharmaceutical composition according to any of the preceding embodiments, wherein said composition is for oral administration.
27. The pharmaceutical composition according to any one of the preceding embodiments, which is in the form of a tablet, a capsule or a mini-tablet.
28. The pharmaceutical composition according to any one of the preceding embodiments, to the extent possible, which is in the form of a tablet.
29. The pharmaceutical composition according to any one of the preceding embodiments, which is in the form of a capsule.
30. The pharmaceutical composition according to any one of the preceding embodiments, to the extent possible, which is in the form of a mini-tablet.
31. The pharmaceutical composition according to any of the preceding embodiments, wherein said GLP-1 receptor agonist is present in the amount of about 0.5 to 60 mg.
32. The pharmaceutical composition according to any of the preceding embodiments, wherein said GLP-1 receptor agonist is present in the amount of about 2.5 to 50 mg.
33. The pharmaceutical composition according to any of the preceding embodiments, wherein said GLP-1 receptor agonist is present in the amount of about 5 to 40 mg.
34. The pharmaceutical composition according to any of the preceding embodiments, wherein said GLP-1 receptor agonist is present in the amount of about 10 to 20 mg.
35. The pharmaceutical composition according to any of the preceding embodiments, wherein said GLP-1 receptor agonist is present in the amount of about 5 mg.
36. The pharmaceutical composition according to any of the preceding embodiments, wherein said GLP-1 receptor agonist is present in the amount of about 10 mg.
37. The pharmaceutical composition according to any of the preceding embodiments, wherein said GLP-1 receptor agonist is present in the amount of about 20 mg.
38. The pharmaceutical composition according to any of the preceding embodiments, to the extent possible, wherein said GLP-1 receptor agonist is present in the amount of about 30 mg.
39. The pharmaceutical composition according to any of the preceding embodiments, to the extent possible, wherein said GLP-1 receptor agonist is present in the amount of about 40 mg.
40. The pharmaceutical composition according to any of the preceding embodiments, to the extent possible, wherein said GLP-1 receptor agonist is present in the amount of about 50 mg.
41. The pharmaceutical composition according to any of the preceding embodiments, to the extent possible, wherein said GLP-1 receptor agonist is present in the amount of about 60 mg.
42. The pharmaceutical composition according to any one of the preceding embodiments, wherein said salt of a medium-chain fatty acid is present in the amount of about 50-700 mg.
43. The pharmaceutical composition according to any one of the preceding embodiments, wherein said salt of a medium-chain fatty acid is present in the amount of about 50-550 mg.
44. The pharmaceutical composition according to any one of the preceding embodiments, to the extent possible, wherein said salt of a medium-chain fatty acid is present in the amount of about 150-600 mg.
45. The pharmaceutical composition according to any one of the preceding embodiments, to the extent possible, wherein said salt of a medium-chain fatty acid is present in the amount of about 180-550 mg.
46. The pharmaceutical composition according to any one of the preceding embodiments, wherein said salt of a medium-chain fatty acid is present in the amount of about 180 mg.
47. The pharmaceutical composition according to any one of the preceding embodiments, to the extent possible, wherein said salt of a medium-chain fatty acid is present in the amount of about 400 mg.
48. The pharmaceutical composition according to any one of the preceding embodiments, to the extent possible, wherein said salt of a medium-chain fatty acid is present in the amount of up to about 700 mg.
49. The pharmaceutical composition according to any of the preceding embodiments, wherein 0.5% (w/w) stearic acid is present.
50. The pharmaceutical composition according to any of the preceding embodiments, wherein 22% (w/w) sorbitol is present.
51. The pharmaceutical composition according to any one of the preceding embodiments, wherein said immediate release coating is present in an amount of about 0 mg/cm2 relative to the tablet core.
52. The pharmaceutical composition according to any one of the preceding embodiments, to the extent possible, wherein said immediate release coating is present in an amount of about 2.6 mg/cm2 relative to the tablet core.
53. The pharmaceutical composition according to any one of the preceding embodiments, to the extent possible, wherein said immediate release coating is present in an amount of about 3.5 mg/cm2 relative to the tablet core.
54. The pharmaceutical composition according to any one of the preceding embodiments, to the extent possible, wherein said immediate release coating is present in an amount of about 4.4 mg/cm2 relative to the tablet core.
55. The pharmaceutical composition according to any one of the preceding embodiments, to the extent possible, wherein said immediate release coating is present in an amount of about 7.1 mg/cm2 relative to the tablet core.
56. The pharmaceutical composition according to any one of the preceding embodiments, to the extent possible, wherein said immediate release coating is present in an amount of about 8 mg/cm2 relative to the tablet core.
57. The pharmaceutical composition according to any one of the preceding embodiments, to the extent possible, wherein said immediate release coating is present in an amount of about 8.8 mg/cm2 relative to the tablet core.
58. The pharmaceutical composition according to any one of the preceding embodiments, to the extent possible, wherein said immediate release coating is present in at amount of about 10.6 mg/cm2 relative to the tablet core.
59. The pharmaceutical composition according to any one of the preceding embodiments, to the extent possible, wherein said immediate release coating is present in at amount of about 11.4 mg/cm2 relative to the tablet core.
60. The pharmaceutical composition according to any one of the preceding embodiments, to the extent possible, wherein said immediate release coating is present in at amount of about 17.6 mg/cm2 relative to the tablet core.
61. The pharmaceutical composition according to any one of the preceding embodiments, to the extent possible, wherein said tablet core weighs between 3.0 and 800 mg.
62. The pharmaceutical composition according to any one of the preceding embodiments, to the extent possible, wherein said tablet core weighs up to 1000 mg.
63. The pharmaceutical composition according to any one of the preceding embodiments, to the extent possible, wherein said tablet core weighs up to 800 mg.
64. The pharmaceutical composition according to any one of the preceding embodiments, to the extent possible, wherein said tablet core weighs about 710 mg.
65. The pharmaceutical composition according to any one of the preceding embodiments, to the extent possible, wherein said tablet core and said immediate release coating weighs about 728 mg.
66. The pharmaceutical composition according to any one of the preceding embodiments, to the extent possible, wherein the weight of the tablet is in the range of 175 mg to 1000 mg.
67. The pharmaceutical composition according to any one of the preceding embodiments, to the extent possible, wherein the weight of the tablet is in the range of 200 mg to 900 mg.
68. The pharmaceutical composition according to any one of the preceding embodiments, to the extent possible, wherein the weight of the tablet is in the range of 500-900 mg.
69. The pharmaceutical composition according to any one of the preceding embodiments, to the extent possible, wherein the weight of the tablet is in the range of 500-700 mg.
70. The pharmaceutical composition according to any one of the preceding embodiments, to the extent possible, wherein the weight of the tablet is in the range of 300-500 mg.
71. The pharmaceutical composition according to any one of the preceding embodiments, to the extent possible, wherein the weight of the tablet is in the range of 175-250 mg.
72. The pharmaceutical composition according to any one of the preceding embodiments, to the extent possible, wherein the weight of the tablet is about 200 mg.
73. The pharmaceutical composition according to any one of the preceding embodiments, to the extent possible, wherein the weight of the tablet is about 400 mg.
74. The pharmaceutical composition according to any one of the preceding embodiments, to the extent possible, wherein the weight of the tablet is about 700 mg.
75. The pharmaceutical composition according to any one of the preceding embodiments, to the extent possible, wherein the weight of the tablet is about 800 mg.
76. The pharmaceutical composition according to any one of the preceding embodiments, to the extent possible, wherein said tablet core, when in the form of a mini-tablet weighs up to 175 mg.
77. The pharmaceutical composition according to any one of the preceding embodiments, to the extent possible, wherein said tablet core, when in the form of a mini-tablet weighs about 4 mg.
78. The pharmaceutical composition according to any one of the preceding embodiments, to the extent possible, wherein said tablet core, when in the form of a mini-tablet and said immediate release coating weighs about 4.3 mg.
79. The pharmaceutical composition according to any one of the preceding embodiments, to the extent possible, wherein the weight of the mini-tablet is in the range of 3 mg to 200 mg.
80. The pharmaceutical composition according to any one of the preceding embodiments, to the extent possible, wherein the weight of the mini-tablet is in the range of 5-15 mg.
81. The pharmaceutical composition according to any one of the preceding embodiments, to the extent possible, wherein the weight of the mini-tablet is in the range of 15-50 mg.
82. The pharmaceutical composition according to any one of the preceding embodiments, to the extent possible, wherein the weight of the mini-tablet is in the range of 50-150 mg.
83. The pharmaceutical composition according to any one of the preceding embodiments, to the extent possible, wherein the weight of the mini-tablet is in the range of 120-160 mg.
84. The pharmaceutical composition according to any one of the preceding embodiments, to the extent possible, wherein the weight of the mini-tablet is about 8 mg.
85. The pharmaceutical composition according to any one of the preceding embodiments, to the extent possible, wherein the weight of the mini-tablet is about 12 mg.
86. The pharmaceutical composition according to any one of the preceding embodiments, to the extent possible, wherein the weight of the mini-tablet is about 30 mg.
87. The pharmaceutical composition according to any one of the preceding embodiments, to the extent possible, wherein the weight of the mini-tablet is about 40 mg.
88. The pharmaceutical composition according to any one of the preceding embodiments, to the extent possible, wherein the weight of the mini-tablet is about 70 mg.
89. The pharmaceutical composition according to any one of the preceding embodiments, to the extent possible, wherein the weight of the mini-tablet is about 100 mg.
90. The pharmaceutical composition according to any one of the preceding embodiments, to the extent possible, wherein the weight of the mini-tablet is about 140 mg.
91. The pharmaceutical composition according to any one of the preceding embodiments, which is in the form of a multiparticulate system.
92. The pharmaceutical composition according to any one of the preceding embodiments, in the form of a multiparticulate system, wherein said particles in said system are individually or collectively coated with said immediate release coating.
93. The pharmaceutical composition according to any one of the preceding embodiments, which is in the form of a uniform tablet, a single or multilayered tablet, a multiparticulate system, a capsule, a tablet contained in a capsule, multiple tablets contained in a capsule, multiple tablets contained in a tablet, a multiparticulate system in the form of a tablet contained in a capsule or in a form of multiparticulate system compressed in one, some or all layers of said tablet core.
94. The pharmaceutical composition according to any one of the preceding embodiments, wherein said GLP-1 peptide is selected from the group consisting of:
N-{Epsilon-26}-[[2-(2-[2-(2-[2-(2-[4-(17-Carboxyheptadecanoylamino)-4(S)-carboxybutyrylamino]ethoxy)ethoxy]acetylamino)ethoxy]ethoxy)acetyl][Aib8,Arg34]GLP-1-(7-37)peptide.

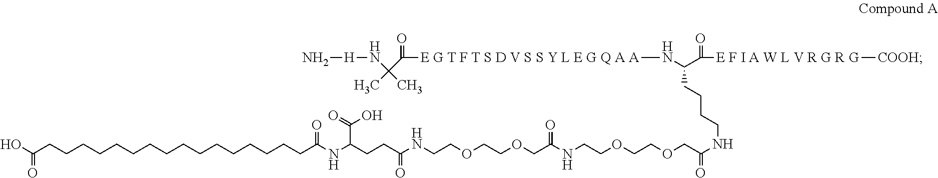

N-{Epsilon-26}-[(2-[2-(2-{2-[2-(2-{(S)-4-Carboxy-4-[10-(4-carboxyphenoxy)decanoylamino]butyrylamino}ethoxy)ethoxy]acetylamino}ethoxy)ethoxy]acetyl}, N-{Epsilon-37}-{2-[2-(2-{2-[2-(2-{(S)-4-carboxy-4-[10-(4-carboxyphenoxy)decanoylamino]butyrylamino}ethoxy)ethoxy]acetylamino}ethoxy)ethoxy]acetyl}-[Aib8,Arg34,Lys37]GLP-1(7-37)-peptide

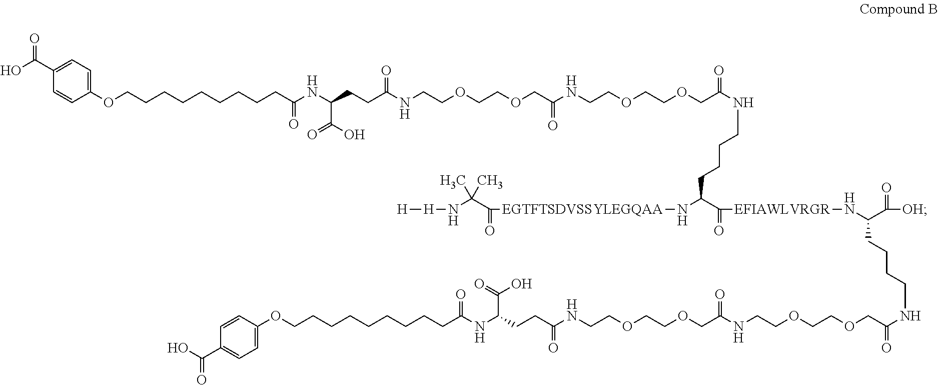

N-{Epsilon-27}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl], N-{Epsilon-36}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib8,Glu22,Arg26,Lys27, Glu30,Arg34,Lys36]-GLP-1-(7-37)-peptidyl-Glu-Gly

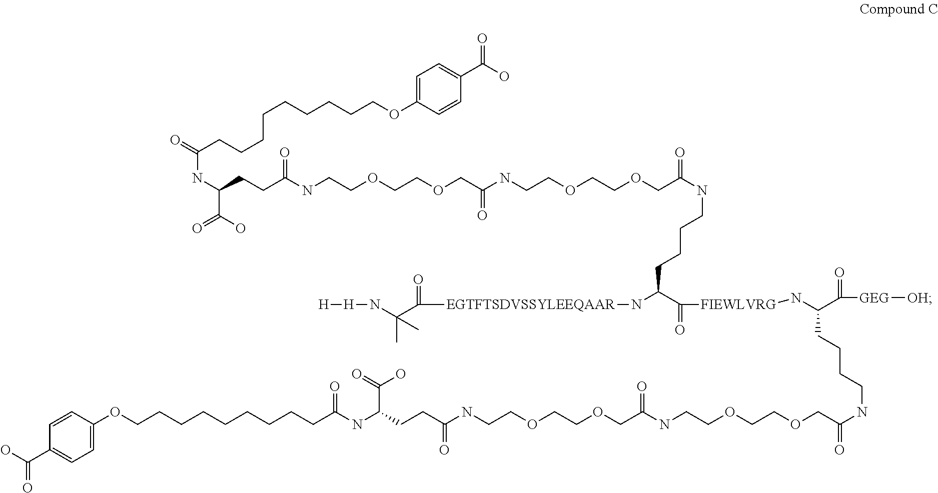

N{Epsilon-36}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl],N{Epsilon-37}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl]-[Aib8,Glu22,Arg26,Arg34,Lys36,Lys37]-GLP-1-(7-37)-peptide

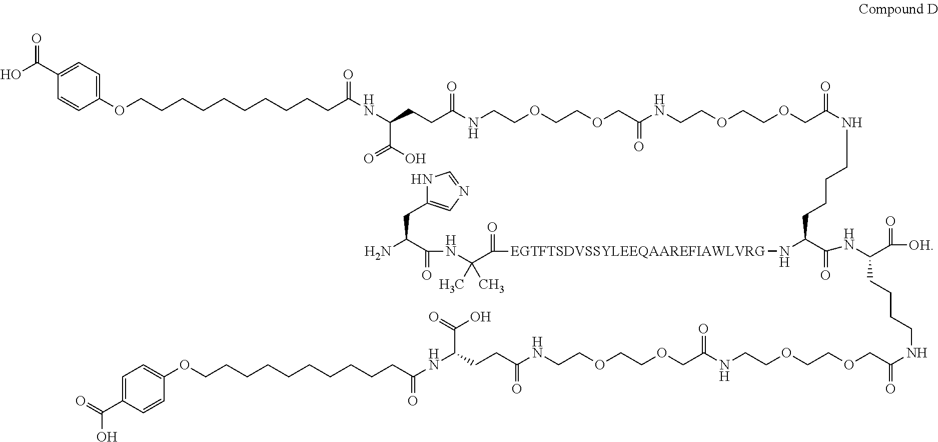

and
N{Epsilon-36}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl],N{Epsilon-37}-[2-[2-[2-[[2-[2-[2-[[(4S)-4-carboxy-4-[11-(4-carboxyphenoxy)undecanoylamino]butanoyl]amino]ethoxy]ethoxy]acetyl]amino]ethoxy]ethoxy]acetyl]-[Aib8,Arg26,Arg34,Lys36,Lys37]-GLP-1-(7-37)-peptide

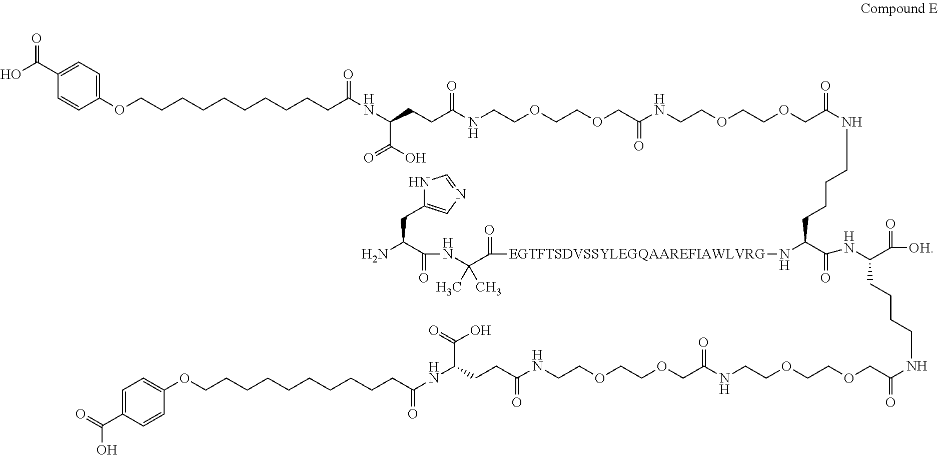

95. The pharmaceutical composition according to any one of the preceding embodiments, wherein said GLP-1 peptide is N{Epsilon-36}-[2-[2-[2-[[2-[2-[2-[[(4S)-4-carboxy-4-[10-(3-carboxyphenoxy)decanoylamino]butanoyl]amino]ethoxy]ethoxy]acetyl]amino]ethoxy]ethoxy]acetyl], N{Epsilon-37}-[2-[2-[2-[[2-[2-[2-[[(4S)-4-carboxy-4-[10-(3-carboxyphenoxy)decanoylamino]butanoyl]amino]ethoxy]ethoxy]acetyl]amino]ethoxy]ethoxy]acetyl]-[Aib8,Glu22,Arg26,Arg34,Lys36,Lys37]-GLP-1-(7-37)-peptide

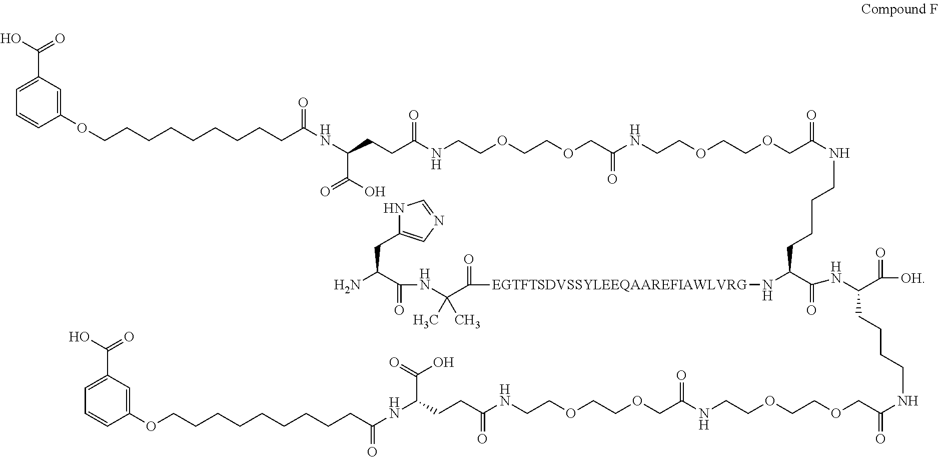

96. The pharmaceutical composition according to any one of the preceding embodiments, wherein said GLP-1 receptor agonist is selected from the group consisting of Compound A, Compound B, Compound C, Compound D, Compound E, and Compound F.
97. The pharmaceutical composition according to any one of the preceding embodiments, wherein said GLP-1 receptor agonist is selected from the group consisting of Compound A, Compound B, Compound C, and Compound F.
98. The pharmaceutical composition according to any one of the preceding embodiments, wherein said GLP-1 receptor agonist is selected from the group consisting of Compound A, Compound B and Compound C.
99. A pharmaceutical composition according to any one of the preceding embodiments, for use as a medicament.
100. The pharmaceutical composition according to the preceding embodiments, for use in preventing and/or treating hyperglycemia, type 2 diabetes, impaired glucose tolerance, type 1 diabetes, non-insulin dependent diabetes, MODY (maturity onset diabetes of the young), gestational diabetes, and/or for reduction of HbA1C.
101. The pharmaceutical composition according to the preceding embodiments, for use in preventing and/or treating diabetes mellitus.
102. The pharmaceutical composition according to the preceding embodiments, for use as a medicament for the treatment of obesity, for preventing overweight, for decreasing food intake and for reducing body weight.
103. The pharmaceutical composition according to the preceding embodiments, for use as a medicament for the treatment of obesity.
104. A method for producing a pharmaceutical composition according to any one of the previous embodiments, comprising the steps of preparing a tablet core and directly coating said immediate release coating on the outer surface of the tablet core.
105. The method for producing a pharmaceutical composition according to any one of the previous embodiments, comprising the steps of preparing a tablet core pressed into a tablet form and directly coating said immediate release coating on the outer surface of said tablet core.

### EXAMPLES

### List of Abbreviations

βAla is beta-alanyl,
Aoc is 8-aminooctanoic acid,
tBu is tert-butyl,
CV is column volumes,
DCM is dichloromethane,
DIC is diisopropylcarbodiimide,
DIPEA=DIEA is N,N-disopropylethylamine,
DMF is N,N-dimethylformamide,
DMSO is dimethyl sulphoxide,
EtOAc is ethyl acetate,
Fmoc is 9-fluorenylmethyloxycarbonyl,
γGlu is gamma L-glutamyl,
HCl is hydrochloric acid,
HOBt is 1-hydroxybenzotriazole,
NMP is N-methylpyrrolidone,
MeCN is acetonitrile,
OEG is [2-(2-aminoethoxy)ethoxy]ethylcarbonyl,
Su is succinimidyl-1-yl=2,5-dioxo-pyrrolidin-1-yl,
OSu is succinimidyl-1-yloxy=2,5-dioxo-pyrrolidin-1-yloxy,
RPC is reverse phase chromatography,
RT is room temperature,
TFA is trifluoroacetic acid,
THF is tetrahydrofuran,
TNBS is 2,4,6-trinitrobenzenesulfonic acid,
TRIS is tris(hydroxymethyl)aminomethane
TSTU is O—(N-succinimidyl)-1,1,3,3-tetramethyluronium tetrafluoroborate.

### Materials and Methods

Method 1: Preparing a Tablet Core According to this Invention

The tablets according to this invention are prepared so that a person skilled in the art of pharmaceutical tablet production easily can make the tablets. The formulation of a tablet core material according to the present invention was performed as outlined here, this example concerns formulations of the present invention comprising:

| GLP-1 peptide | 1.4 or 0.7% | (w/w) |
|---|---|---|
| Sodium salt of capric acid | 77.5% | (w/w) |
| Sorbitol | 20.6 or 21.3% | (w/w) |
| Stearic acid | 0.5% | (w/w) |

wherein the amount of sorbitol depends on the amount of GLP-1 peptide and is 22-X % (w/w) sorbitol, where X is the amount of GLP-1 peptide (in % w/w).

When 100 g of tablet core material comprising GLP-1, sodium caprate (sodium salt of capric acid), sorbitol and stearic acid was manufactured according to the above listed ingredients and in the corresponding ratios, the following steps were used:

Sorbitol powder was sieved using a mesh size of 0.5 mm. After sieving, the correct amount was weighed.

GLP-1 peptide and sorbitol were mixed in a small container. An amount of sorbitol equivalent to the amount of GLP-1 peptide was added to the container and the ingredients were mixed manually. Then the double amount of sorbitol relative to the previous addition was added and the ingredients were mixed manually until GLP-1 peptide and all sorbitol were mixed well. This step was followed by a mechanical mixing in a Turbula-mixer to finalize the mixing to obtain a homogeneous powder.

Sodium salt of capric acid (in the form of granulate) was then added to the GLP-1 peptide-sorbitol powder according to equal volumes principle. This was done in two steps and finalized with a mechanical mixing step in a Turbula-mixer.

Finally stearic acid was sieved using a mesh size of 0.25 mm. Stearic acid was weighed and added to the powder and the ingredients were mixed mechanically.

Method 2: Preparing a Tablet Core with an Immediate Release Coat, Such as Opadry®II Yellow

The powder prepared according to method 1 was compressed in a tablet press to form tablets of a mass of 710 mg. A tablet core prepared by this method was then coated with immediate release coating comprising polyvinyl alcohol (PVA). The coating solution was prepared by dispersing the 20 g immediate release coating material comprising PVA and/or HPMC in 80 g demineralised water. The concentration of immediate release coating in the coating solution was 20%-(w/w). Under intense mixing using a standard magnetic stirrer the polymer powder was added to the water. After addition of polymer the mixture was stirred at low intensity for 30 minutes. The resulting coating solution was sieved to remove lumps. The coating of tablet cores was performed in a pan coater with the pan size of 8.5″, with a conventional patterned air Schlick spray nozzle with an orifice of 1.0 mm, an atomizing and pattern air pressure of 0.5-0.6 bar, inlet air temperature of 35-36° C. and air flow of 95-100 kg/hour. The coating was performed by pumping the polymer solution in through the nozzle. After addition of ex 4.5% (w/w) dry weight of the polymer powder distributed evenly on the tablet cores the spraying was stopped and the tablets were allowed to dry for up to 30 minutes inside the pan.

Method 3: Preparing a Tablet Core with an Immediate Release Coat, SUCH AS OPADRY® Clear

The powder prepared according to method 1 was compressed in a tablet press to form tablets of a mass of 710 mg. A tablet core prepared by this method was then coated with immediate release coating comprising hydroxypropylmethylcellulose (HPMC). The coating solution was prepared by dispersing the 15 g immediate release coating material comprising HPMC in 85 g demineralised water. The concentration of immediate release coating in the coating solution was 15% (w/w). Under intense mixing using a standard magnetic stirrer the polymer powder was added to the water. After addition of polymer the mixture was stirred at low intensity for 30 minutes. The resulting coating solution was sieved to remove lumps. The coating of tablet cores was performed in a pan coater with the pan size of 8.5″, with a conventional patterned air Schlick spray nozzle with an orifice of 1.0 mm, an atomizing and pattern air pressure of 0.5-0.6 bar, inlet air temperature of 40° C. and air flow of 95-100 kg/hour. The coating was performed by pumping the polymer solution in through the nozzle. After addition of ex 2.2% (w/w) dry weight of the polymer powder distributed evenly on the tablet cores the spraying was stopped and the tablets were allowed to dry for up to 30 minutes inside the pan.

Method 4: Preparing Mini-Tablets with an Immediate Release Coat, Such as Opadry®II Yellow

The powder prepared according to method 1 was compressed into mini-tablets on a tablet press. Coating of mini-tablets was performed using a fluid bed apparatus equipped with a Wurster insert (mini-Glatt®, as sold in 2014) via the following steps:

Preparation of the Coating Solution:

for the preparation of 100 g of coating solution, 20 g of Opadry® II Yellow were dispersed in 80 g demineralised water. The suspension was stirred using a standard magnetic stirrer for 30 minutes, and afterwards sieved to remove eventual lumps. The suspension was kept under stirring during the coating process.

Coating:

Coating of mini-tablets was performed in a fluid bed apparatus equipped with a Wurster insert (mini-Glatt®, as sold in 2014). The fluid bed chamber was pre-heated until a temperature of 30-35° C. inside the chamber was reached. An accurately weighed amount of mini-tablets (20 g) was placed in the fluid bed chamber and warmed up for 2 min or until they reached 30° C. in temperature. Spray layering was performed by pumping the solution through a nozzle with an orifice of 0.8 mm, at an atomising pressure of 0.9 bar. The inlet air temperature, within the range 50-55° C., was adjusted throughout the process to keep the product temperature at 30-35° C. Coating was stopped when a coating level of 4 mg/cm2 (equivalent to a weight gain of 26%) was reached.

Drying of Mini-Tablets:

mini-tablets were dried in the same equipment at 50° C. for 3 min A weighed mass of 710 mg mini-tablets was manually filled into gelatine capsules.

### Method 5: In Vivo Pharmacokinetic Studies in Beagle Dogs

The day before the experiment the Beagle dogs were weighed and fed their normal diet at 12 pm and was given an overnight fast with ad libitum access to water. On the day of the experiment the dogs were placed on a test platform and fitted with a 20G Venflon in v. cephalica to allow for blood sampling. During the first 2.5-4 h the blood samples were collected from the Venflon after which time the Venflon was removed and the dogs returned to their pens. For subsequent blood sampling the dogs were lead into a test room and a blood sample was taken from the v. jugularis using a standard 21G needle and a syringe. This procedure was also employed when it was not possible to place a venflon in v. cephalica. In a subset of studies the dogs remained in their pens also in the first 4 h and were lead to a test room were all blood samples were taken from the v. jugularis using a standard 21G needle and a syringe. The gycA dogs were fed 4 h after dosing.

### IV Dosing

The dogs were positioned on the test platform and after placement of the venflon in v. cephalica the GLP-1 peptide was administered IV in v. cephalica of the other front leg by using a 23G butterfly needle. After the GLP-1 peptide was administered the bufferfly was flushed using physiological saline containing 10 IU/mL of heparin. In some studies, dogs were not on platforms and fitted with a venflon but remained in their pens and were dosed directly into the v. cephalica.

### Standard Oral Administration of Tablets

The dogs were positioned on the test platform and after placement of the venflon the tablet was administered in the following manner: The tablet was placed in the back of the mouth of the dog in order to prevent chewing. The mouth was closed and approx 10 mL of tap water was given by a syringe to facilitate swallowing of the tablet. In some studies the dogs were not on platforms but were dosed when they were still in the pens.

Oral Administration after Subcutaneous (SQ) Pentagastrin Injection

In a subset of studies gastric acid secretion was induced before administration of the oral tablet. Pentagastrin was administered subcutaneously at a dose of 4 μg/kg body weight (120 μg/mL) was administered 20 minutes prior to the per os (PO or per oral administration) dose.

### Blood Sampling

The following applies to all types of studies described herein.

Before each blood sample was collected, the first few drops were allowed to drain from the venflon to avoid saline from the venflon in the sample. For each time point ˜800 μL of whole blood was collected in a 1.5 mL EDTA coated tube, and the tube was gently turned to allowing mixing of the sample with the anticoagulant. The samples were placed on ice until centrifugation at 4000G (4° C.) for 4 min, and afterwards pipetted on dry ice into micronic tubes for later analysis of GLP-1 peptide. All samples were kept at −80° C. until plasma analysis.

Blood samples were collected to adequately cover the full plasma concentration-time profile of the GLP-1 peptide. For example blood samples were collected at the following times: (t) predose, 0.25 hour, 0.5 hour, 0.75 hour, 1 hour, 1.5 hours, 2 hours, 4 hours, 6 hours, 8 hours, 10 hours, 24 hours, 48 hours, 72 hours, 120 hours, 144 hours, 168 hours, 192 hours, 216 hours, 240 hours, and 288 hours after dosing.

After each blood sample the Venflon was flushed using 0.5 mL saline containing heparin (10 IU/mL).

All plasma samples were analyzed using either sandwich immunoassay (LOCI) or

Liquid chromatography-mass spectrometry.

Pharmacokinetic Profile

Increased terminal half-life (T½) or decreased clearance from the blood results in a longer period where the GLP-1 peptide may exert its pharmacological effect, meaning that the tested GLP-1 peptide is eliminated slower from the body than with a shorter terminal half-life.

The pharmacokinetic (PK) profile of a GLP-1 peptide of a pharmaceutical composition of the present invention may be suitably be determined by in-vivo PK studies. These studies were performed in order to evaluate how the GLP-1 peptide was absorbed, distributed and eliminated from the body and how these processes affected the plasma concentration-time profile of the GLP-1 peptide.

In discovery and preclinical phase of drug development numerous methods and animal models may be utilized to understand the PK properties for the active pharmaceutical ingredient (API). In the current invention the beagle dog was used exclusively to evaluate the PK of the GLP-1 peptide of the pharmaceutical composition of the invention following oral administration.

From the GLP-1 peptide measurements in the blood samples individual plasma-time profile were plotted and the data were analyzed by non-compartmental pharmacokinetics (NCA) using Phoenix WinNonlin 6.3 (Pharsight In., Mountain View, Ca, USA).

Many compounds will have a 1st order elimination in the terminal phase of the plasma concentration-time profile and hence show linearity when depicted on a semi-logarithmic plot. Consequently, after the initial absorption and distribution of the GLP-1 peptide it will be eliminated from the body at a constant fractional rate. This rate was determined as elimination rate constant (Ke) and calculated as—slope of linear terminal phase. From Ke also the plasma T½ was calculated as T½=ln(2)/Ke. From the non-compartmental analysis the area under the plasma concentration-time profile (AUC) was determined by using the trapezoidal rule and linear-log extrapolation from last measurement point to infinity (Johan Gabrielsson and Daniel Weiner: Pharmacokinetic and Pharmacodynamic Data Analysis. Concepts & Applications, 3rd Ed., Swedish Pharmaceutical Press, Stockholm (2000)).

### Method 6: Oral Bioavailability in Dogs

Increased oral bioavailability means that a larger fraction of the dose administered orally reach the systemic circulation from where it can distribute to exhibit pharmacological effect.

Generally, the term bioavailability refers to the fraction of an administered dose of active pharmaceutical ingredient (API), that reaches the systemic circulation unchanged. By definition, when an API is administered intravenously, its bioavailability is 100%. However the API can be incompletely absorbed following oral administration, or be degraded either within the intestinal lumen or in first pass hepatic metabolism. Knowledge of absolute bioavailability (F) is necessary when designing dosage regiments for oral administration.

Herein, a plasma concentration-time plot was made and using NCA, the dose-corrected AUC was calculated after both oral administration and intravenous administration to beagle dogs, specifically F was calculated as AUC/Dₚₒ divided by AUC/Dᵢᵥ.

### Example 1: Preparation and Composition of Tablets Comprising Compound A

Tablets comprising N{Epsilon-26}-[2-[2-[2-[[2-[2-[2-[[(4S)-4-carboxy-4-(17-carboxy-heptadecanoylamino)butanoyl]amino]ethoxy]ethoxy]acetyl]amino]ethoxy]ethoxy]acetyl]-[Aib8,Arg34] GLP-1(7-37)-peptide as the GLP-1 peptide were prepared.

A tablet core with 1.4% (w/w) GLP-1 peptide, 77.5% (w/w) Sodium decanoate/Sodium salt of capric acid, 20.6% (w/w) Sorbitol and 0.50% (w/w) Stearic acid was prepared using method 1. The tablet was compressed and optionally coated using method 2 or 3.

Tablets were administered per os to Beagle dogs according to method 5.

Bioavailability (F, calculated as AUC/Dₚₒ relative to AUC/Dᵢᵥ) and Tmax (determined as the time when the plasma concentration of GLP-1 peptide reached its maximum level, ie. the time of observed Cmax) were determined. The results are shown in table 1 with bioavailability shown as mean values.

TABLE 1

| Coating | Bioavailability (%) | Tmax (hours) |
|---|---|---|
| Uncoated | 1.0 | 1.8 hr |
| 2.5% Opadry ® II Yellow | 0.8 | 1.6 hr |
| 4.5% Opadry ® II Yellow | 0.5 | 2.1 hr |
| 5% Opadry ® II Yellow | 0.5 | 1.4 hr |
| 2.2% Opadry ® Clear | 1.0 | 2.5 hr |

### Example 2: Preparation and Composition of Tablets Comprising Compound B

Tablets comprising N{Epsilon-26}-[{2-[2-(2-{2-[2-(2-{(S)-4-Carboxy-4-[10-(4-carboxyphenoxy)decanoylamino]butyrylamino}ethoxy)ethoxy]acetylamino}ethoxy)ethoxy]acetyl}, N{Epsilon-37}-[{2-[2-(2-{2-[2-(2-{(S)-4-carboxy-4-[10-(4-carboxyphenoxy)decanoylamino]butyrylamino}ethoxy)ethoxy]acetylamino}ethoxy)ethoxy]acetyl}-[Aib⁸,Arg³⁴,Lys³⁷]GLP-1 (7-37)—OH as the GLP-1 peptide were prepared.

A tablet core with 1.4% (w/w) GLP-1 peptide, 77.5% (w/w) Sodium decanoate/Sodium salt of capric acid, 20.6% (w/w) Sorbitol and 0.50% (w/w) Stearic acid was prepared using method 1. The tablet was compressed and optionally coated using method 2 or 3.

Tablets/capsules were administered per os to Beagle dogs according to method 5.

Bioavailability (F, calculated as AUC/Dₚₒ relative to AUC/Dᵢᵥ) and Tmax (determined as the time when the plasma concentration of GLP-1 peptide reached its maximum level, ie. the time of observed Cmax) were determined. The results are shown in table 2 with bioavailability shown as mean values.

TABLE 2

| | | Tmax |
|---|---|---|
| Coating | Bioavailability (%) | (hours) |
| Opadry 4.5% + AcrylEZE 93A 11.6% | 0.4 | 4.0 |
| (PO adm. after SQ pentagastrin injection) | | |
| Uncoated | 2.6 | 1.3 |
| 2.5% Opadry ® II Yellow | 4.4 | 1.1 |
| 4.5% Opadry ® II Yellow | 3.3 | 1.1 |
| 2.5% Opadry ® Clear | 3.3 | 1.0 |
| 2.5% Opadry ® II Yellow | 3.7 | 2.0 |
| mini-tablets in a gelatine capsule | | |

### Example 3: Preparation and Composition of Tablets Comprising Compound C

Tablets comprising N{Epsilon-27}-[[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl], N{Epsilon-36}-[[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib⁸,Glu²²,Arg²⁶,Lys²⁷,Glu³⁰,Arg³⁴,Lys³⁶]-GLP-1-(7-37)-peptidyl-Glu-Gly as the GLP-1 peptide were prepared.

A tablet core with 0.7% (w/w) GLP-1 peptide, 77.5% (w/w) Sodium decanoate/Sodium salt of capric acid, 21.3% (w/w) Sorbitol and 0.50% (w/w) Stearic acid was prepared using method 1. The tablet was compressed and coated using method 2.

Tablets were administered per os to Beagle dogs according to method 5.

Bioavailability (F, calculated as AUC/Dₚₒ relative to AUC/Dᵢᵥ) and Tmax (determined as the time when the plasma concentration of GLP-1 peptide reached its maximum level, ie. the time of observed Cmax) were determined. The results are shown in table 3 with bioavailability shown as mean values.

TABLE 3

| Coating | Bioavailability (%) | Tmax (hours) |
|---|---|---|
| 2.5% Opadry ® II Yellow | 4.5 | 1.5 |

### Example 4: Preparation and Composition of Tablets Comprising Compound D

Tablets comprising Compound D as the GLP-1 peptide are prepared.

A tablet core with 0.7% (w/w) GLP-1 peptide, 77.5% (w/w) Sodium decanoate/Sodium salt of capric acid, 21.3% (w/w) Sorbitol and 0.50% (w/w) Stearic acid are prepared using method 1. The tablet is compressed and coated using method 2.

Tablets are administered per os to Beagle dogs according to method 5.

Bioavailability (F, calculated as AUC/Dpo relative to AUC/Div) and Tmax (determined as the time when the plasma concentration of GLP-1 peptide reached its maximum level, i.e. the time of observed Cmax) are determined.

### Example 5: Preparation and Composition of Tablets Comprising Compound E

Tablets comprising Compound E as the GLP-1 peptide are prepared.

A tablet core with 0.7% (w/w) GLP-1 peptide, 77.5% (w/w) Sodium decanoate/Sodium salt of capric acid, 21.3% (w/w) Sorbitol and 0.50% (w/w) Stearic acid are prepared using method 1. The tablet is compressed and coated using method 2.

Tablets are administered per os to Beagle dogs according to method 5.

Bioavailability (F, calculated as AUC/Dpo relative to AUC/Div) and Tmax (determined as the time when the plasma concentration of GLP-1 peptide reached its maximum level, i.e. the time of observed Cmax) are determined.

### Example 6: Preparation and Composition of Tablets Comprising Compound F

Tablets comprising Compound F (i.e. N{Epsilon-36}-[2-[2-[2-[[2-[2-[2-[[(4S)-4-carboxy-4-[10-(3-carboxyphenoxy)decanoylamino]butanoyl]amino]ethoxy]ethoxy]acetyl]amino]ethoxy]-ethoxy]acetyl], N{Epsilon-37}-[2-[2-[2-[[2-[2-[2-[[(4S)-4-carboxy-4-[10-(3-carboxyphenoxy)-decanoylamino]butanoyl]amino]ethoxy]ethoxy]acetyl]amino]ethoxy]ethoxy]acetyl]-[Aib8,Glu22,Arg26,Arg34,Lys36,Lys37]-GLP-1-(7-37)-peptide as the GLP-1 peptide were prepared.

A tablet core with 0.7% (w/w) GLP-1 peptide, 77.5% (w/w) Sodium decanoate/Sodium salt of capric acid, 21.3% (w/w) Sorbitol and 0.50% (w/w) Stearic acid was prepared using method 1. The tablet was compressed and coated using method 2.

Tablets were administered per os to Beagle dogs according to method 5.

Bioavailability (F, calculated as AUC/Dpo relative to AUC/Div) and Tmax (determined as the time when the plasma concentration of GLP-1 peptide reached its maximum level, i.e. the time of observed Cmax) were determined. The results are shown in Table 6 with bioavailability shown as mean values.

TABLE 6

| Coating | Bioavailability (%) | Tmax (hours) |
|---|---|---|
| 2.5% Opadry ® II Yellow | 2.3 | 1.5 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pharmaceutical composition comprising a tablet core and an immediate release coating, wherein said tablet core comprises an absorption enhancer and a GLP-1 receptor agonist.

2. The pharmaceutical composition according to claim 1, wherein said immediate release coating is the outer layer of said pharmaceutical composition.

3. The pharmaceutical composition according to claim 1, wherein said immediate release coating dissolves in aqueous medium at any pH.

4. The pharmaceutical composition according to claim 1, wherein said immediate release coating is a polyvinyl alcohol based coating and/or a hydroxypropyl-methylcellulose based coating.

5. The pharmaceutical composition according to claim 4, wherein said immediate release coating is selected from Opadry®, Opadry®Clear, Opadry®II, Opadry®II Clear, Opadry®II Pigmented or Opadry®II Yellow.

6. The pharmaceutical composition according to claim 5, wherein said immediate release coating is selected from OPADRY®II Yellow or OPADRY® clear.

7. The pharmaceutical composition according to claim 1, wherein said absorption enhancer is a salt of a medium-chain fatty acid.

8. The pharmaceutical composition according to claim 7, wherein said salt of a medium-chain fatty acid is sodium caprate.

9. The pharmaceutical composition according to claim 1, wherein said tablet core further comprises one or more excipients.

10. The pharmaceutical composition according to claim 1, wherein said GLP-1 receptor agonist is human GLP-1, exendin-4 or an analogue or derivative thereof.

11. The pharmaceutical composition according to claim 10, wherein said GLP-1 receptor agonist is selected from the group consisting of Compound A, Compound B, Compound C, Compound D, Compound E and Compound F.

12. The pharmaceutical composition according to claim 1, wherein said composition is for oral administration.

13. The pharmaceutical composition according to claim 1, wherein said composition is in the form of a tablet, a mini-tablet or a capsule.

14. (canceled)

15. A method for producing a pharmaceutical composition according to claim 1, comprising the steps of preparing a tablet core and directly coating said immediate release coating on the outer surface of the tablet core.

16. The pharmaceutical composition according to claim 7, wherein said salt of a medium-chain fatty acid is present in the amount of about 50-700 mg.

17. A method of treating or preventing hyperglycemia, type 2 diabetes, impaired glucose tolerance, type 1 diabetes or obesity, preventing overweight, decreasing food intake or reducing body weight, comprising administering the pharmaceutical composition according to claim 1 to a patient in need thereof.
